(19) United States
(12) Patent Application Publication
(10) Pub. No.: US 2022/0211308 A1
(43) Pub. Date: Jul. 7, 2022

(54) APPARATUS FOR MEASURING BIOLOGICAL FUNCTION, A METHOD AND A PROGRAM FOR MEASURING BIOLOGICAL FUNCTION

(71) Applicant: Toshinori KATO, Tokyo (JP)

(72) Inventor: Toshinori KATO, Tokyo (JP)

(21) Appl. No.: 17/445,142

(22) Filed: Aug. 16, 2021

Related U.S. Application Data
(63) Continuation of application No. PCT/JP2019/005721, filed on Feb. 15, 2019.

(51) Int. Cl. A61B 5/1455; A61B 5/00

(52) U.S. Cl. CPC A61B 5/14553; A61B 5/7239; A61B 5/742

(57) ABSTRACT

Problem To provide an apparatus for evaluating biological function, and a method and a program for evaluating biological function, for the purpose of measuring and quantifying biological function, to make possible the quantification of measured values concerning brain characteristics at time of rest.

Means for resolution The apparatus for evaluating biological function (1) is [an apparatus] that utilizes the near-infrared spectroscopy method to evaluate biological function; measuring part 5 computes time course change amounts of oxyhemoglobin and time course change amounts of deoxyhemoglobin, based on light information from detecting parts 4; and it has a computing part 8, which obtains a group of zeroset vectors at prescribed sampling times based on 2-dimensional diagrams showing the relationship between change amounts of oxyhemoglobin and change amounts of deoxyhemoglobin, and computes parameters based on the directions and/or scalars of that vector group.

### TECHNICAL FIELD

The present invention relates an apparatus for measuring biological function, a method and a program for evaluating biological function, for the purpose of measuring and evaluating the function of a living body; and in particular, it relates an apparatus for evaluating biological function, a method and a program for evaluating biological function for measuring and evaluating the function of a living body in order to make possible the quantification of measurements during a period wherein a task for activating brain function is not assigned to the living body (below, “at rest”).

### BACKGROUND IN THE ART

As for prior art, a method was advocated by F. F. Jobsis in 1977, in which the brain is irradiated with weak near-infrared rays (680-1300 nanometers) through the skull from outside the scalp to measure concentration change amounts of oxyhemoglobin (OxyHb, HbO₂) and concentration change amounts of deoxyhemoglobin (DeoxyHb, Hb) in blood in the brain surface (cerebral cortex) just inside the skull. Since that time, research on the measurement of tissue oxygen concentration by means of this near-infrared spectroscopy (NIRs) method has progressed rapidly.

In general, the near-infrared spectroscopy method has the advantages that an individual's tissue metabolism can be measured noninvasively from the surface of the body (noninvasiveness); it can be implemented by a simple and convenient apparatus (portability); in addition, unlike PET (positron emission tomography), f-MRI (functional magnetic resonance imaging) and the like, measurements of changes in tissue metabolism over time of the brain, muscles, and the like can be obtained in real time (temporality); and a wide range of applications have been anticipated in areas of practical use such as brain function monitoring, evaluation of muscle rehabilitation in physical therapy, and exercise physiology.

The conventional Jobsis method undertakes to monitor brain oxygen noninvasively, and an optical tomography method (optical CT), in which the brain was cross-sectioned in layers by straight-line light, was devised in an attempt to obtain accurate oxygen saturation information to the depths of the brain, (Shinohara, Y. et al., Optical CT imaging of hemoglobin oxygen-saturation using dual-wavelength time gate technique. Adv Exp Med Biol, 1993. 333: p. 43-6).

However, even if accurate location information could have been measured by the technique of optical CT, by the time the light passed to the brain surface, through the skull and into the brain, it was absorbed and was of no practical use.

Accordingly, in 1991, the present inventor Kato proposed and corroborated new basic principle of NIRS imaging (near-infrared spectroscopy brain functional imaging method) to determine location information by means of probe location and the response of a measurement target on the brain surface.

In addition, the present inventor and colleagues performed human experiments of photic stimulation, in which the brain was partially irradiated with near-infrared light, and as a result, they showed that distribution of localized brain function can be monitored at the bedside, and proved that, using a bedside noninvasive detection method with this method, images can be created of localized brain function (Sachio Takashima, Toshinori Kato, et al., “NIR Spectroscopy ni yoru kyokusho nouketsuryu hendou no kansatsu”. Shinshingaiji(sha) no iryou ryouiku ni kansuru sougouteki kenkyu no houkokusho [“Observation of local brain blood flow variation by means of NIR spectroscopy”, in Comprehensive Research Report Concerning Medical Care for Children (People) with Disabilities] (Japan Ministry of Health and Welfare), p. 179-181 (1992); Kato T, Kamei A, et al., “Human visual cortical function during photic stimulation monitoring by means of near-infrared spectroscopy”, J Cereb Blood Flow Metab. 13:516-520 (1993).)

These basic principles of near-infrared spectroscopy brain functional imaging (NIRS imaging) are currently utilized in, for example, technique[s] for graphically displaying the functional topography of the brain surface in the frontal region, the occipital region and the like (hemoglobin distribution maps; namely, the display of increases and decreases in blood volume, reflecting brain activity, as topographical maps), and in pioneering techniques for obtaining brain activation information.

Conventionally, with NIRS, it was possible to perform noninvasive monitoring of changes in degree of oxygen saturation, changes in brain blood volume and the like.

As methods for simultaneously measuring changes in oxygen metabolism occurring when the brain is activated, the techniques of Patent Reference 1, Patent Reference 2, and Patent Reference 3, have been proposed, as inventions devised by the present inventor.

From the use of changes in localized ratios of OxyHb and DeoxyHb and an index obtained from 2-dimensional diagrams, a new index of brain function was born. In addition, brain activity can be demarcated by means of phase change information obtained from changes of localized ratios of OxyHb and DeoxyHb.

By means of these inventions, also as brain functional imaging methods, changes of degree of oxygen saturation and changes in blood volume could be distinguished, and temporal and spatial mapping became possible. Currently, it has also become possible to detect from outside the scalp a signal called the initial dip, which is associated with neural activity. From (Toshinori Kato [Nov. 5, 2018]. Vector-based Approach for the Detection of Initial Dips Using Functional Near-Infrared Spectroscopy [Working Title], IntechOpen, DOI:10.5772/intecechoen.80888. Available from: https://www.intechopen.com/online-first/vector-based-approach-for-the-detection-of-initial-dips-using-functional-near-infrared-spectroscopy/), and the techniques of Patent Reference 3, it became possible to measure localized OxyHb and DeoxyHb change amounts, which were until then measured as time course data, as change amounts with respect to distance moved. (Referred to below as “Previous Example 1”.)

In addition, techniques focused on brain changes at rest and brain changes during activation have been proposed. For example, in Patent Reference 4, a brain activation estimating apparatus for estimating human brain activity is disclosed, having a blood circulation volume calculation part wherein, based on RGB data of photographic image data obtained by means of RGB processing performed on photographic image data of a human face acquired in time series, the color components are broken down into the three components of R, G and B, to calculate blood circulation volume of said face time series; and an estimating part, based on a plurality of components obtained by breaking down the blood circulation data by means of singular value decomposition, principal component analysis, or independent component analysis, for estimating brain activity of said person; and from the plurality of components, the means for estimating human brain activity extracts a component as the determination component, whose component wave form amplitude correlates with changes between brain rest and brain activation, and estimates human brain activity based on the determination component.

With this previous apparatus, the period in which said brain functional activation task is not assigned to the human is taken as the time when the brain is at rest, and the time when said brain functional activation task is assigned to said human is taken as the time when the brain is active, and said plurality of components are evaluated as to whether or not said correlation is present. (Referred to below as “Previous Example 2”.)

In addition, as a technique for quantitatively investigating the brain of a living body at rest, in Patent Reference 5, an apparatus for measuring degree of stress has been proposed, making it possible to quantitatively grasp the degree of stress at a time of stress load with respect to a time of rest.

In Patent Reference 6, an activation brain wave monitoring method and an activation brain wave monitoring apparatus are proposed to quantitatively investigate brain wave changes during and after hyperventilation, in which they are displayed as power spectra, with brain wave forms at rest, before hyperventilation, as the standard; and frequency analysis on induced brain waves is performed at prescribed intervals, power values of an arbitrary frequency range are calculated and the rate of change of power values of a prescribed frequency range is displayed as percentages on a trend graph taking brain waves before activation as the standard. (Referred to below as “Previous Example 3”.)

Patent Reference 1: Japan patent No. 4031438

Patent Reference 2: Japan patent No. 4625809

Patent Reference 3: Japan patent No. 6029236

Patent Reference 4:

Japan patent publication No. 2017-209516

Patent Reference 5:

Japan patent publication No. 1996-126614

Patent Reference 6:

Japan patent publication No. 2000-279388

### DISCLOSURE OF THE INVENTION

### Problem[s] To Be Solved by the Invention

Previous Example 1, had the following kinds of problems.

(1) When ratio k and angle k were utilized, in the case of making phase divisions, because phase division was dependent on signal strength, very small valid activity was difficult to detect. From only signal strength, correct and highly precise evaluation was difficult.

(2) In the case of measuring a plurality of sites, even comparison between channels was dependent on signal strength.

(3) There were cases in which brain functional task A or brain functional task B had to be quantified, compared to time at rest, but they were compared by taking the difference between task A and task B. For that reason, it was not possible to quantify the state at rest.

(4) Even after a task was finished, it was undefined whether or not the brain had returned to a resting state.

(5) Because of the phenomenon of a blood flow increase occurring and high oxidation after low oxidation when oxygen has been used in the tissues, it frequently happened that blood flow was increasing even after a task, and it wasn't possible to tell if it was a resting state or an after-effect of the task.

(6) When the brain does nothing, quantitative evaluation at rest has been difficult. Different types and roles of the nerve cell groups of the brain can be considered to be divided into “addresses” according to their location, showing different situation even at rest. In the case of fNIRS, the start time is taken as zero, and relative changes are captured after a prescribed time passes. However, the state at rest was not at all discernable.

(7) In some cases, numbers of statistically significant channels have been counted, ignoring locality.

(8) In Previous Example 1, after setting an arbitrary point to zero, the amounts of change of each hemoglobin have been recorded as relative change amounts. Namely, after “zero” was set, because they are relative change amounts, quantification of “at rest” was not possible. With NIRS measurements according to the continuous wave method known as CW, because the optical path length cannot be measured, cases of quantitative measurements became estimates.

(9) In cases in which a plurality of sites were measured using a multi-channel NIRS apparatus, even if the optical path length of each site differed, they were assumed to be the same, and concentration changes for each hemoglobin were mapped. On the other hand, when the optical path length is measured, using methods such as time-resolved spectroscopy (TRS)and phase-resolve spectroscopy (PRS), it would take several minutes at rest, and thus quantification of the resting state in real time by changes in milliseconds or changes in meters was not possible. In fact, with TRS, measuring brain oxygen saturation of one location at rest from on the scalp requires approximately 5 minutes.

(10) The total optical path length can be measured using methods such as TRS and PRS. However, in cases of changes in the state at rest, because partial optical path lengths of sites where blood flow changes accompany brain activity cannot be surveyed, it has been difficult to quantitatively calculate local hemoglobin concentration changes.

(11) With NIRS measurements, because signal amplitudes differ according to the location of irradiation and light-receiving fiber pairs, for NIRS signal amplitude comparisons between sites and between individuals, it has been difficult to compare greater and lesser blood flow responses.

(12) Even at rest, because the baselines of time series change data of each hemoglobin or migration length correspondence data may change greatly; or, filters may have been applied as baseline corrections to large fluctuations of amplitude; or, because analysis processing, such as migration averaging, smoothing, linear functions or quadratic functions may have been done arbitrarily, actual phase information may have been deformed, or [the data] may have been utilized as is with an analysis bias that arose in the actual data at rest.

(13) The cerebral hemodynamic frequency is considered to be 0.1 Hz or less, and because low-pass filters have been commonly used, and because high frequency changes have been generally considered to be noise, it has not been possible to tell whether high frequency region changes are resting state or active state.

In Previous Example 2, nothing is disclosed regarding techniques for making possible the quantification of measured values in time of rest, and nothing is recited to suggest it.

Patent Reference 5 of Previous Example 3 is a technique for quantifying the degree of stress corresponding to an amount of finger skin temperature reduction. Because it detects the response of a part of the body, it assumes that the brain receives stress, and as a result, a reaction appears also in a part of the body, such as the skin. Because types of neural cells differ by brain site and their function also differs, as stress received by a brain site having to do with the skin, for example, the sensory area[s], that could a reasonable explanation in some cases. However, stress received by other sites, outside the sensory area, such as the motor system, comprehension system, visual system, thought system, etc., cannot be discerned and detected by this method.

Patent Reference 6 of Previous Example 3 is a technique for quantitatively investigating brain waves that takes brain wave waveforms at rest before hyperventilation as standard, and displays their changes during and after hyperventilation. In this technique, quantification is possible by site from on the scalp. However, it requires frequency analysis at prescribed intervals, and for this reason, changes from moment to moment are missed, and even if there are differences in the time courses by site, it's possible they are missed and [the sites] are viewed as being the same.

The present invention was done for the purpose of solving the problems described above, and takes as its objective the provision of an apparatus for evaluating biological function, and a method and a program for evaluating biological function, for the purpose of measuring and evaluating the function of a living body, to enable the quantification of measured values concerning brain characteristics at time of rest.

### Means for Solving the Problems

The apparatus for evaluating biological function of the present invention is:

an apparatus for evaluating biological function that evaluates biological function utilizing the near-infrared spectroscopy method, having a plurality of detecting parts provided with light-emitting parts for irradiating light to a prescribed site of a living body, and light-receiving parts for receiving and detecting light emanating from within the living body; a measuring part, into which light information detected by means of said detecting parts is input, and wherein computation, control and/or memory operations are performed; and a determination part, which determines the state of biological function of said living body;

and it is characterized in that said measuring part has a computing means that computes time course change amounts of oxyhemoglobin and time course change amounts of deoxyhemoglobin based on light information from said detecting parts, obtains a group of vectors zero-set for each prescribed sampling time, based on 2-dimensional diagrams showing the relationship between said change amounts of oxyhemoglobin and change amounts of deoxyhemoglobin, and computes parameters based on the directions and/or scalars of said vector group;

and said determination part determines the state of biological function based on said parameters, computed by means of said computing means.

The aforementioned parameters may be dynamic phase division ratios, based on said 2-dimensional diagrams, showing how frequently the vectors of said vector group appear in the plurality of phase divisions into which it is divided.

The aforementioned parameters may be ones concerning vector norms of said vector group.

Said parameters may be any or all of: average vector norms, dispersion of norms, standard deviations of norms, of said vector group, computed using circular statistics (angular statistics).

Said parameters may be parameters computed based on a probability density function of a Rayleigh distribution.

Said parameter may be parameters concerning correlation properties of 2 orthogonal axial directions on said 2-dimensional diagrams.

Said parameters may also concern dual-axis correlations of said group of vectors.

Said parameters may also be parameters computed using derivatives of time course change amounts of oxyhemoglobin and time course change amounts of deoxyhemoglobin.

Said parameters may be parameters computed using derivatives of said time course change amounts of oxyhemoglobin and time course change amounts of deoxyhemoglobin, that are differentiated a plurality of times.

Said parameters may be parameters computed varying said sampling times.

Said parameters may be parameters computed selecting a number of unit width points which are said sampling time×n (where n is an arbitrary number such that n>1).

Values of said parameters may be displayed on a display part, with data plotted on said 2-dimensional diagrams.

Said determination part may take the period when there is no task for activating brain function assigned to said living body to be brain time at rest, and determine the state of said living body function in that time at rest.

Said phase division diagrams may be divided into eight divisions.

Said phase division diagrams may be divided into 24 divisions.

The method for evaluating biological function of the present invention is

a method for evaluating biological function performed by means of an apparatus for evaluating biological function wherein biological function is evaluated utilizing the near-infrared spectroscopy method, having a plurality of detecting parts provided with light-emitting parts for irradiating light to a prescribed site of a living body, and light-receiving parts for receiving and detecting light emanating from the living body; a measuring part, into which light information detected by means of said detecting parts is input, and computation, control and/or memory operations are performed; and a determination part, which determines the state of biological function of said living body;

and it is characterized in that it is one having a step whereby time course change amounts of oxyhemoglobin and time course change amounts of deoxyhemoglobin are computed, based on light information from said detecting parts; and

a step whereby a group of vectors zero-set for each prescribed sampling time is obtained based on 2-dimensional diagrams showing the relationship between said change amounts of oxyhemoglobin and change amounts of deoxyhemoglobin, and parameters are computed, based on the directions and scalars of said vector group; and

a step whereby the state of biological function is determined based on said computed parameters.

The program of the present invention is

a program of the present invention for implementing a biological function evaluation process performed by means of an apparatus for evaluating biological function that evaluates biological function utilizing the near-infrared spectroscopy method, having a plurality of detecting parts provided with light-emitting parts for irradiating light to a prescribed site of a living body, and light-receiving parts for receiving and detecting light emanating from the living body; a measuring part, into which light information detected by means of said detecting parts is input, and wherein computation, control and/or memory operations are performed; and a determination part, which determines the state of biological function of said living body;

and it is characterized in that it is one that implements a process whereby the time course change amounts of oxyhemoglobin and time course change amounts of deoxyhemoglobin are computed, based on light information from said detecting part;

a process whereby amounts of time course changes of oxyhemoglobin concentration and amounts of time course changes of deoxyhemoglobin concentration are computed, based on light information from said detecting part;

and processes for obtaining a group of vectors zero-set for each prescribed sampling time, based on a 2-dimensional diagram showing the relationship between said change amounts of oxyhemoglobin and change amounts of deoxyhemoglobin, and for computing parameters, based on the directions and scalars of said vector group;

and a process whereby the state of biological function is determined, based on said computed parameters.

### Effect of the Invention

The present invention exhibits the following effects.

(1) The sensitivity of real-time neurofeedback, brain-computer interface (BCI), and brain-machine interface (BMI) can be improved.

(2) It becomes a new index of brain function, making quantitative mapping possible.

(3) The state of the brain at rest can be quantified by site. With conventional techniques, it was thought that all sites were in the same state at the measurement start origin when high frequency changes were measured and analyzed.

(4) Both fNIRS and fMRI have been used at rest, but even when correlations between sites were examined, there was no means for quantification by sites measured (measurement of degree of brain oxygen saturation at rest was difficult). In contrast, this became possible with the present invention.

(5) By quantifying the state during sleep, with eyes open/closed, and the like, it becomes possible to differentiate [these states].

(6) Quantification of advancing stages of dementia by means of the state of the brain is possible.

(7) Real-time quantitative measurement of the state at rest is possible, in units of milliseconds or in units of meters. As a result, degree of precision of detection of brain activity can also be improved.

(8) The oxygen state at rest can be quantified without utilizing degree of oxygen saturation.

(9) By brain waves, it was possible to analyze frequency and the like at rest, but there was no way to quantify rest in units of milliseconds; this is possible with the present invention.

(10) Evaluation of the stress state at rest is possible. Furthermore, is it possible to not simply quantify the strength of stress, but detailed classification of the stress state also becomes possible, by computation of average vector norms (R), distribution (V) of norms L, their standard deviation (S), using phase distribution, circular (angular) statistics and the like.

(11) In the case of analysis at prescribed intervals, because it is possible to analyze average vector norms (R), distribution (V) of norms L, their standard deviation (S) not only in the case of values of (ΔOxyHb, ΔDeoxyHb), but also to analyze average vector norms (R), distribution (V) of norms ΔL, and their standard deviations (S) in the case of values (ΔΔOxyHb, ΔΔDeoxyHb), it is possible, compared to power spectrum analysis, to distinguish very minute physiological differences of the resting state, and to distinguish between rest and activation tasks.

(12) By analysis of zeroset vector values (phase and scalar amounts), detailed classification of arbitrary intervals of the state at rest is possible.

(13) Display of reproducibility of the state at rest and during an activation task became possible, as reproducibility rate mapping from APR (Active Phase Rate) values, rotating coordinate system dual-axis correlation coefficients, and the like.

It has become possible to display reproducibility of the state at rest and during a task as reproducibility mapping, from APR (active phase ratio) values and dual-axis correlation coefficients of rotation coordinate systems and the like.

(14) Without a smoothing process in analysis, the fluctuation of the high signal had a strong influence on the direction of the vectors, but significant changes can now be detected without a smoothing process.

(15) Even in cases when simultaneous group measurements of a plurality of subjects are taken, the instant of synchronization can be seen.

(16) A method has become possible in which it is ok not to use a typical brain blood flow model.

(17) Comparison of individuals, tasks and sites can be performed quantitatively.

(18) The measured data of the concentration change of each hemoglobin can be quantified at rest without baseline correction, filter processing, and the like.

(19) It is possible to quantify which axis the fluctuations at rest are approaching most closely on 2-dimensional vector diagrams formed by an oxygen exchange axis (ΔOE) and a blood flow amount axis (ΔBF), and to quantify the phases (angles).

(20) Even if baseline correction at rest is done, it can be implemented by a method that does not distort the true values.

(21) The state at rest can be quantified by different frequency bands.

### A BRIEF DESCIPTION OF THE DRAWINGS

FIG. 1 is a block diagram showing the constitution of the apparatus for evaluating biological function of an embodiment of the present invention.

FIG. 2 is an explanatory view showing a vector model that shows the geometric relationship between the oxygen saturation angle and hemodynamic indices, in which the horizontal axis is concentration change amounts of oxyhemoglobin, and the vertical axis is concentration change amounts of deoxyhemoglobin.

FIG. 3A is a graph for explaining the oxygen saturation angle and the degree of oxygen saturation, in which the horizontal axis is concentration change amounts of oxyhemoglobin, and the vertical axis is concentration change amounts of deoxyhemoglobin; 3B is a graph showing phase segments, in which the horizontal axis is concentration change amounts of oxyhemoglobin, and the vertical axis is concentration change amounts of deoxyhemoglobin; and 3C is a graph showing the relationship between the oxygen saturation angle and the degree of oxygen saturation, in which the horizontal axis is angle of oxygen saturation (degrees) and the vertical axis is degree of oxygen saturation.

FIG. 4A is a graph showing the relationship between degree of oxygen saturation and the oxygen saturation angle, angle Y, in which the horizontal axis is concentration change amounts of oxyhemoglobin, and the vertical axis is concentration change amounts of deoxyhemoglobin; and 4B is a corresponding table of these.

FIG. 5 is a flow chart for explaining the operation of the apparatus for evaluating biological function of an embodiment of the present invention.

FIG. 6 is a graph showing the dynamic phase division ratio computation step, and it is a graph of acquired data with the horizontal axis as time, and the vertical axis as change amounts of oxygenated and deoxygenated hemoglobin.

FIG. 7 is a graph that takes the horizontal axis as time and the vertical axis as change amounts of hemoglobin; it shows the procedure whereby an arbitrary interval is selected, mark widths are set, and zeroset processing is done.

FIG. 8 is a graph that takes the horizontal axis as concentration change amounts of oxyhemoglobin and the vertical axis as concentration change amounts of deoxyhemoglobin; it shows the procedure whereby each of the group of zeroset vectors is classified into a phase division.

FIG. 9. is a graph, before conversion, in which the horizontal axis is time and the vertical axis is change amounts of hemoglobin (oxyhemoglobin, deoxyhemoglobin and total hemoglobin), for showing an example of computation of dynamic phase division ratios.

FIG. 10A is a graph showing the horizontal axis as mark width points (1=75 ms) and the vertical axis as dynamic phase division ratios (Dynamic phase division ratio=APR values) in two phases.

FIG. 10B is a graph for confirming the dependence on each of the mark widths (set time lengths) in 8 phases.

FIG. 11 is a graph for confirming the dependence of mark widths in a case when phases 2, 3, 4, 5 and 6 are taken as active phases and phases 1, 7 and 8 are taken as inactive phases; it is a graph showing the horizontal axis as unit numbers of mark widths (1=75 ms) and the vertical axis as dynamic phase division ratios (APR values).

FIG. 12 is a graph in which time course data is plotted on a 2-dimensional diagram, in which the horizontal axis is concentration change amounts of oxyhemoglobin, and the vertical axis is concentration change amounts of deoxyhemoglobin; [this] is displayed on a display part in the form of a radar chart divided into 8 phase divisions; and the dynamic phase division ratios, which are proportions (%) for each phase, are displayed on the display part.

FIG. 13 is a bar graph showing the dynamic phase division ratios (APR values) in the positive phases 1-5 and the negative phases 6-8.

FIG. 14 is a bar graph that takes the horizontal axis as phases and the vertical axis as dynamic phase division ratios (APR values).

FIG. 15A At the same channel (channel 2), it is a graph in which computations are done for an arbitrary 8-second interval at rest; and 15B is a graph at the same channel (channel 14) for a task in which, in an interval of 0-3 seconds, a subject hears the word “lion” and responds “lion”, repeating this eight times; and for that time, at the measurement site, for a total of 8 seconds, from 2 seconds before the word “lion” is said until the subject finishes speaking, average oxyhemoglobin change amounts, average deoxyhemoglobin change amounts, and the time band[s] showing active phases (AP) are computed and compared, and displayed as a time course.

FIG. 16A At the same channel (channel 2), it is a graph in which computations are done for an arbitrary 8-second interval at rest; and 16B is a graph at the same channel (channel 16), for a task in which, in an interval of 0-3 seconds, [a subject] hears the word “lion” and responds “lion” repeating this eight times; and for that time, at the measurement site, for a total of 8 seconds, from 2 seconds before the word “lion” is said until the subject finishes speaking, average oxyhemoglobin change amounts, average deoxyhemoglobin change amounts, and the time bands showing active phases (AP) are computed and compared, and displayed as time courses.

FIG. 17 At the same channel, it is a graph in which, in 8 trials at ch15, in a case in which during an interval of 0-3 seconds a subject heard the word “lion” and responded “lion”; and for a total of 8 s, including 2 s before the hearing interval and 3 s after the speech interval, average oxyhemoglobin change amounts, average deoxyhemoglobin change amounts, and average dynamic phase division ratios (average APR values) (%) were computed and compared and displayed as time courses.

FIG. 18 At the same channel, it is a graph in which, in 8 trials at ch15, in a case in which during an interval of 0-3 seconds a subject heard the word “lion” and responded “lion”; and for a total of 8 s, including 2 s before the hearing interval and 3 s after the speech interval, average oxyhemoglobin change amounts, average deoxyhemoglobin change amounts and average angles k were computed and compared and displayed as time courses.

FIG. 19 At the same channel, it is a graph in which, in 8 trials at channel ch15, in a case in which during an interval of 0-3 seconds a subject heard “lion” and responded “lion”; and for a total of 8 s, including 2 s before the hearing interval and 3 s after the speech interval, average oxyhemoglobin change amounts, average deoxyhemoglobin change amounts, and derivatives of angle k, computed from derivatives of average oxyhemoglobin change amounts and derivatives of average deoxyhemoglobin change amounts, were computed and compared, and displayed as time courses.

FIG. 20A is a graph in which the horizontal axis is time (s) and the vertical axis is dynamic phase division ratio (APR values), and 20B is a graph in which the horizontal axis is time (s) and the vertical axis is change amounts of oxyhemoglobin; 46 channels were disposed on the top of the head, and a task was performed in which the subject strongly bit down on something for 3 s with the left masseter muscle, and the results were compared with time at rest; and time course data of change amounts of oxyhemoglobin and active phase ratios (APR) in that time were compared.

FIG. 21 is mapping figures for a task in which a plurality of channels were disposed on the top of the head, and [the subject] strongly bit down on an object for 3 s with the left masseter muscle, and the results were compared with time at rest; 21A is an APR map for a left side masseter muscle task, and 21B is a map of change amounts of oxyhemoglobin for a left side masseter muscle task.

FIG. 22 FIG. 22 is a time course graph in which the subject's measured data at rest shown in FIG. 9 are each differentiated (ΔΔOxyHb, ΔΔDeoxyHb); the horizontal axis is time and the vertical axis is derivatives of concentration change amounts of oxyhemoglobin (ΔΔOxyHb) and derivatives of concentration change amounts of deoxyhemoglobin (ΔΔDeoxyHb), showing their relative changes.

FIG. 23A is a 2-dimensional diagram in which a subject's measured data at rest shown in FIG. 9 (ΔOxyHb, ΔDeoxyHb) are displayed respectively as the horizontal axis and the vertical axis; 23B is a 2-dimensional diagram in which the data of FIG. 22 (ΔΔOxyHb, ΔΔDeoxyHb) are displayed respectively as the horizontal axis and the vertical axis.

FIG. 24 is an explanatory view showing a case in which the phase diagram is divided into 24 divisions.

FIG. 25 is bar graphs in which the horizontal axis is phase (24 divisions) and the vertical axis is dynamic phase division ratios (APR values); 25A is the same data of a subject's state at rest in FIG. 14, there divided into 8 divisions, divided [here] into 24 divisions, and 25B shows the case of the subject while moving.

FIG. 26A is a bar graph in which the horizontal axis is phases (24 divisions) and the vertical axis is dynamic phase division ratios (APR values) at channels ch16, ch17 and ch18 of the brain; 26A shows the subject at rest.

FIG. 26B shows the case of the subject while moving.

FIG. 27A shows a time course graph of original data obtained from channels of the frontal region at rest, displayed with the horizontal axis as time and the vertical axis as concentration change amounts of oxyhemoglobin and concentration change amounts of deoxyhemoglobin. 27B shows a time course graph in which a baseline drift setting of a 0.1 increase, continuing for 5 minutes, is added to both the oxyhemoglobin (OxyHb) and deoxyhemoglobin (DeoxyHb) original data of (A), and displayed.

FIG. 28 shows a time course graph in which a baseline drift setting of a 0.1 increase, continued for 5 minutes, is added to only the oxyhemoglobin (OxyHb) original data of (A) and displayed.

FIG. 29A is a bar graph showing frequency distribution of radius R of (ΔOxyHb, ΔDeoxyHb) in subject A.

FIG. 29B is a bar graph in which the frequency distribution of (ΔΔOxyHb, ΔΔDeoxyHb) shows a Rayleigh distribution.

FIG. 30A is a bar graph showing the frequency distribution of radius R of (ΔOxyHb, ΔDeoxyHb) in subject B.

FIG. 30B is a bar graph in which the frequency distribution of (ΔΔOxyHb, ΔΔDeoxyHb) shows a Rayleigh distribution.

FIG. 31A is a scatter plot graph showing angular radius dispersion of (ΔOxyHb, ΔDeoxyHb), and 31B, angular radius dispersion of (ΔΔOxyHb, ΔΔDeoxyHb).

FIG. 32A is a bar graph in which the horizontal axes are channel numbers (ch) and the vertical axis is dynamic phase division ratios (APR values), which compares data over 224 s, at rest and when moving; 32A is a graph showing norms R (at rest), from which APRs are created for the channels.

FIG. 32B is a graph showing norms R (when moving), from which the APR is created for each channel.

FIG. 32C is a graph showing standard deviations S of norms R (at rest), from which APRs are created for the channels.

FIG. 32D is a graph showing standard deviations S of norms R (when moving), from which APRs are created for the channels.

FIG. 33 is a graph showing a rotating coordinate system at rest (Generalized COE) for computing dual-axis correlation coefficients of coordinate rotation angle θ using a zeroset vector group.

FIG. 34A is a time course graph of Rest Period A obtained from a channel of the frontal region of the head, in which the horizontal axis is time and the vertical axis is concentration change amounts of oxyhemoglobin and concentration change amounts of deoxyhemoglobin; 34B is a time course graph of Rest Period B obtained from a channel of the frontal region of the head, in which the horizontal axis is time and the vertical axis is concentration change amounts of oxyhemoglobin and concentration change amounts of deoxyhemoglobin.

FIG. 35 is a rotating coordinate system dual-axis correlation graph of Rest Period A and Rest Period B, in which the horizontal axis is coordinate rotation angles (degrees) and the vertical axis is dual-axis correlation coefficients, and it shows the relationship between the two.

FIG. 36 is a rotating coordinate system dual-axis correlation graph of measured data obtained from the motor area (M1) during rest and during the exercise of lifting a 9.5 kg dumbbell.

FIG. 37 is a rotating coordinate system dual-axis correlation graph of measured data obtained from a site adjacent to the motor area (M1) during rest and during the exercise of lifting a 9.5 kg dumbbell.

FIG. 38A is a graph displaying the data of FIG. 34A, to which a first-order 0.1 Hz Butterworth low-pass filter was applied; the horizontal axis is time and the vertical axis is concentration change amounts of oxyhemoglobin and concentration change amounts of deoxyhemoglobin. 38B is a graph displaying the data of FIG. 34B, to which a first-order 0.1 Hz Butterworth low-pass filter has been applied; the horizontal axis is time and the vertical axis is concentration change amounts of oxyhemoglobin and concentration change amounts of deoxyhemoglobin.

FIG. 39 is a rotating coordinate system dual-axis correlation graph of rest period (A) and rest period (B) showing the relationship between the coordinate rotation angle and the dual-axis correlation coefficients after low-pass filtering.

FIG. 40A is a graph displaying the data of FIG. 34A, to which a first-order 0.1 Hz Butterworth high-pass filter was applied; the horizontal axis is time and the vertical axis is concentration change amounts of oxyhemoglobin and concentration change amounts of deoxyhemoglobin. 40B is a graph displaying the data of FIG. 34B, to which a first order 0.1 Hz Butterworth high-pass filter was applied; the horizontal axis is time and the vertical axis is concentration change amounts of oxyhemoglobin and concentration change amounts of deoxyhemoglobin.

FIG. 41 is a rotating coordinate system dual-axis correlation graph of Rest Period A and Rest Period B, showing the relationship between coordinate rotation angles and dual-axis coordinate coefficients, after high-pass filtering.

### BEST EMBODIMENT FOR IMPLEMENTATION OF THE INVENTION

An embodiment of the present invention is described below with reference to the drawings.

### Outline of the Apparatus for Evaluating Biological Function

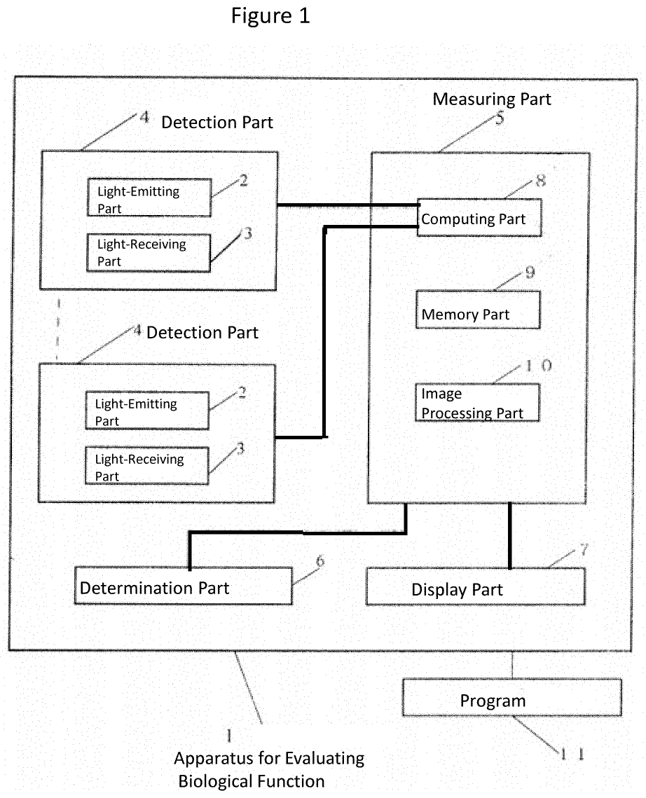

FIG. 1 is a block diagram showing the configuration of an apparatus for evaluating biological function of an embodiment of the present invention.

An apparatus for evaluating biological function 1 of the embodiment of the present invention is one that evaluates biological function utilizing the near-infrared spectroscopy method, and as shown in FIG. 1, it has a plurality of detecting parts 4, provided with light-emitting parts 2, which irradiate light to a prescribed site of a living body, and light-receiving parts 3, which receive and detect light emanating from inside the living body; a measuring part 5, into which light information detected by means of detecting part 4 is entered, and computation, control and/or memory operations are performed; a determination part 6, which determines the state of biological function of said living body; and a display part 7, which is a monitor, display or the like, which displays various data measured by measuring part 5, determination results from determination part 6, and the like.

Measuring part 5 computes time course change amounts of oxyhemoglobin and time course change amounts of deoxyhemoglobin, based on light information from detecting part 4; and, based on 2-dimensional diagrams showing the relationship between change amounts of oxyhemoglobin and change amounts of deoxyhemoglobin, obtains a group of vectors, zeroset at prescribed sampling intervals, and computes parameters based on the directions and/or scalars of that vector group.

Measuring part 5 also has a memory part 9, which stores data computed by means of computing part 8; and an image processing part 10, which creates graphs, tables and the like, based on the computed data, and displays them on display part 7.

Determination part 6 determines the state of living body function based on parameters computed by means of computing part 8.

### Regarding Parameters

Parameters computed by means of computing part 8 include examples such as the following:

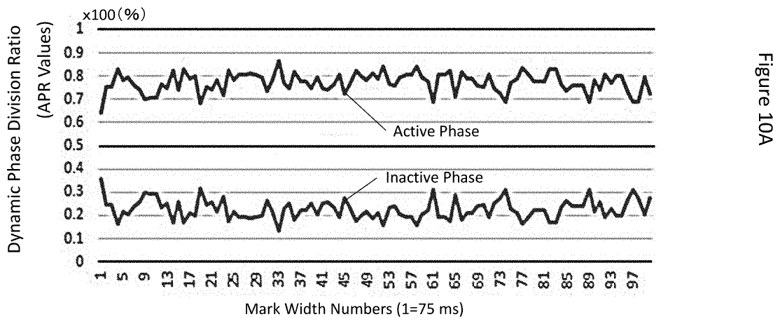

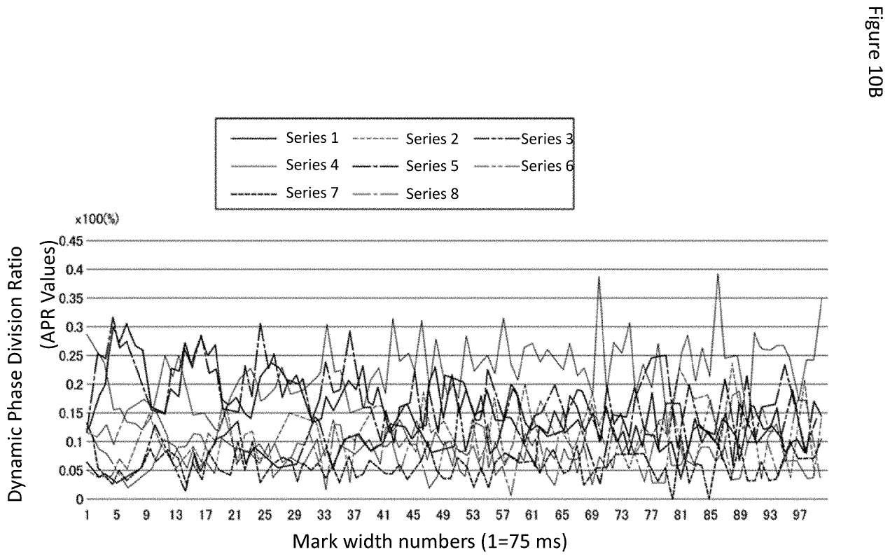

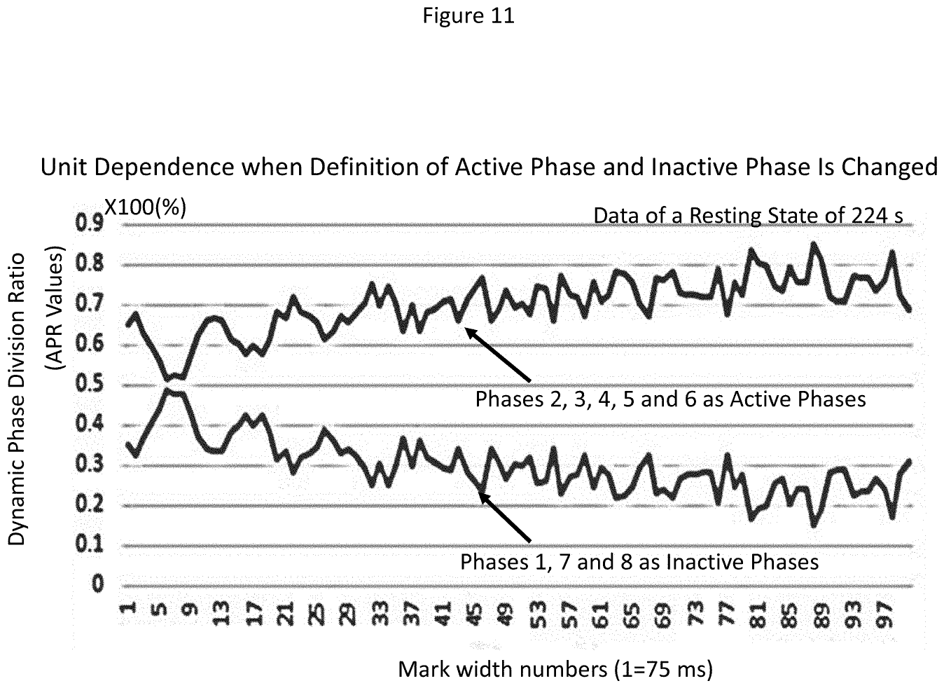

(1) Dynamic phase division ratios (FIGS. 10, 11, etc.), which show, based on 2-dimensional diagrams, how frequently the vectors of the vector group appear in the plurality of phase divisions into which the diagram is divided.

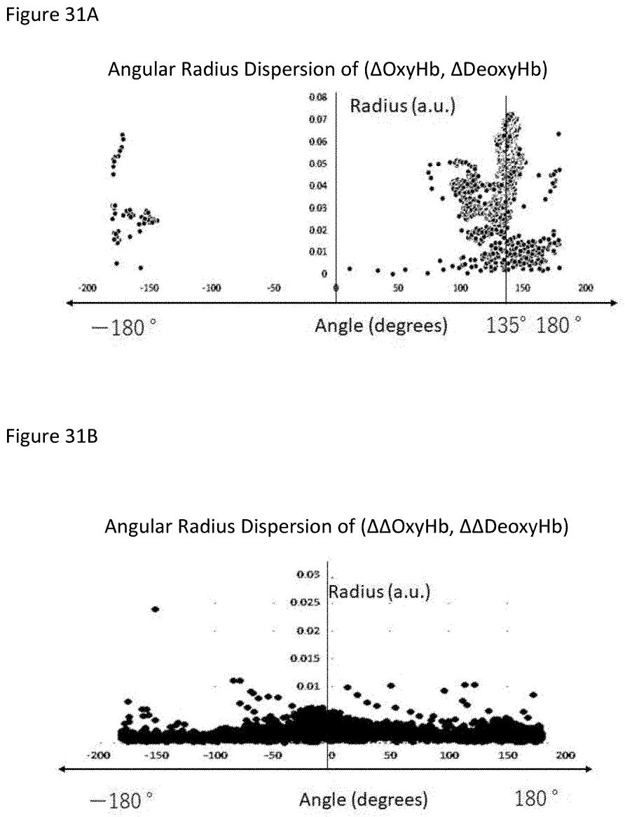

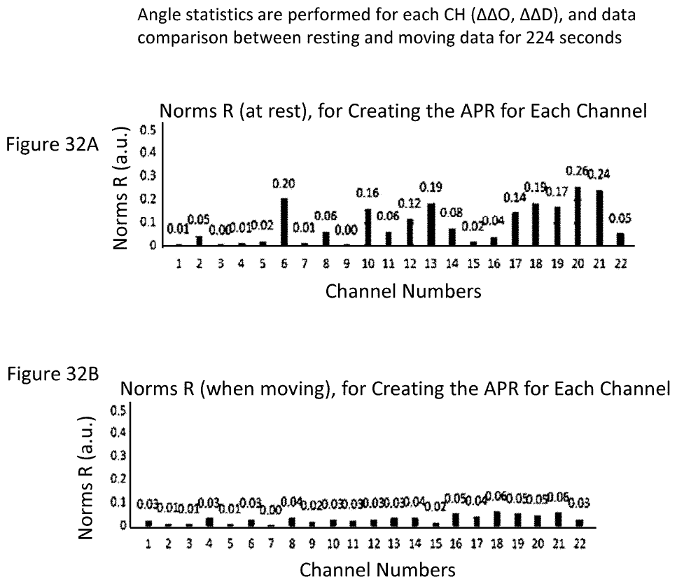

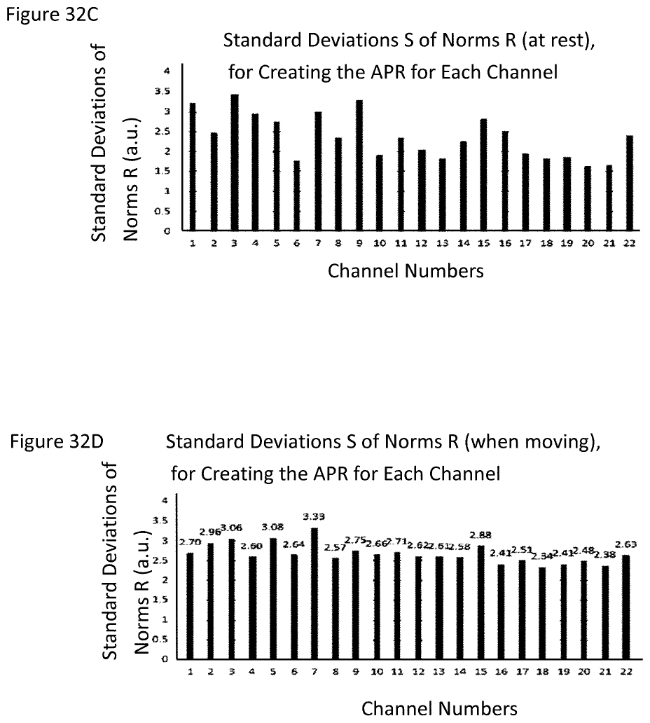

(2) Parameters concerning norms of the vectors of the vector group (FIG. 31, FIG. 32). Here, in the Specification, “scalar”, “norm” and “radius” are used with the same meaning.

(3) Average vector norms, dispersion of norms, standard deviations of norms (FIG. 32), computed using circular (angular) statistics.

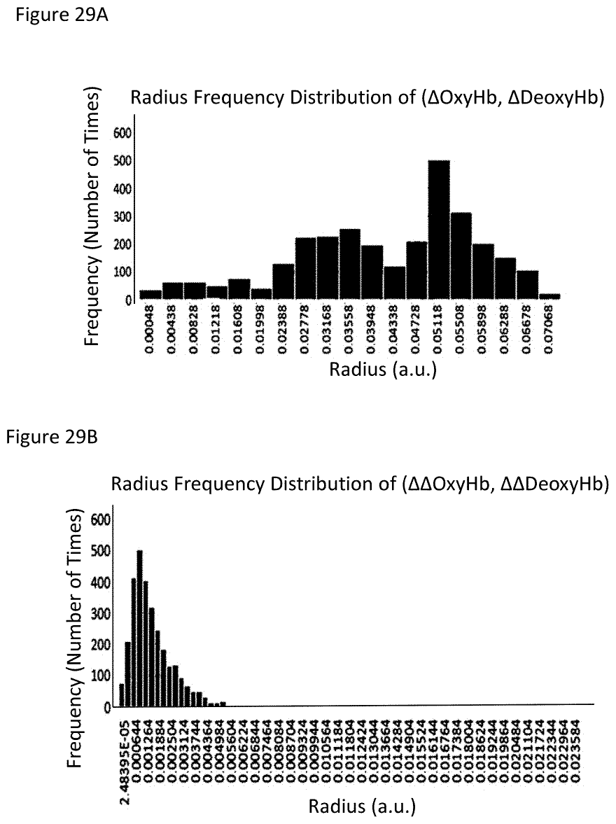

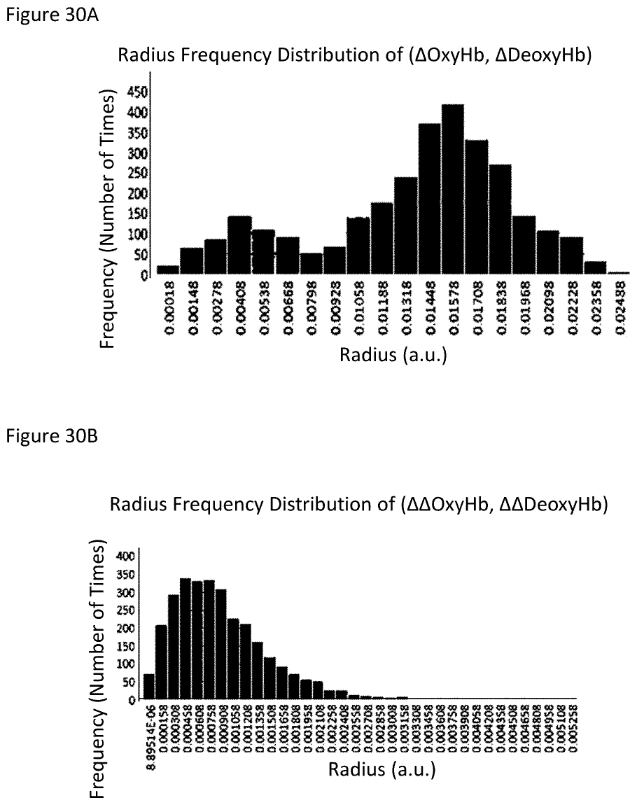

(4) Parameters computed based on the probability density function of a Rayleigh distribution (FIG. 29, FIG. 30).

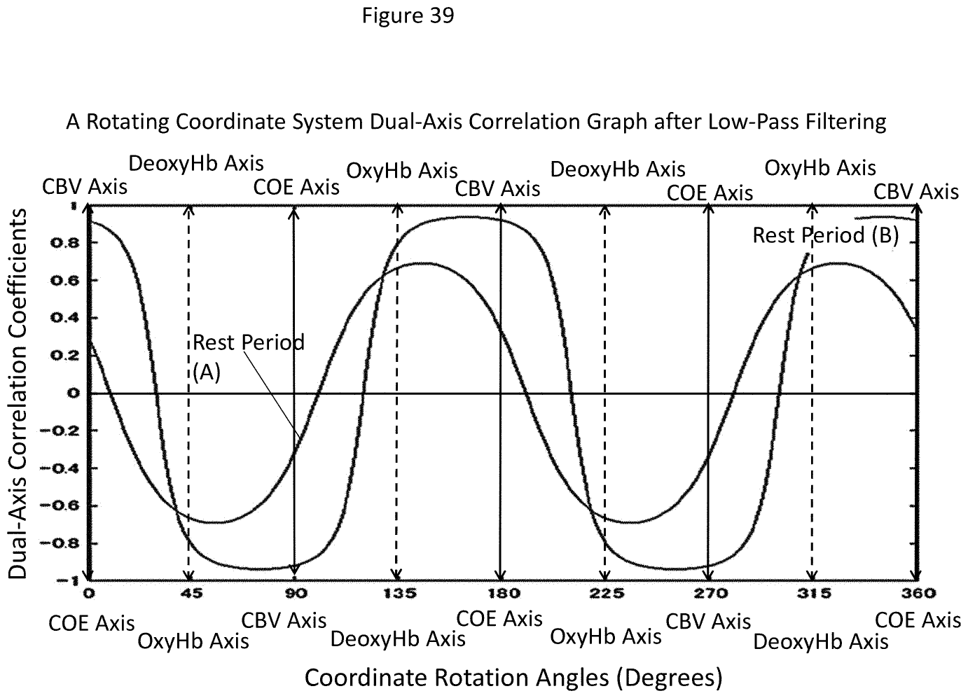

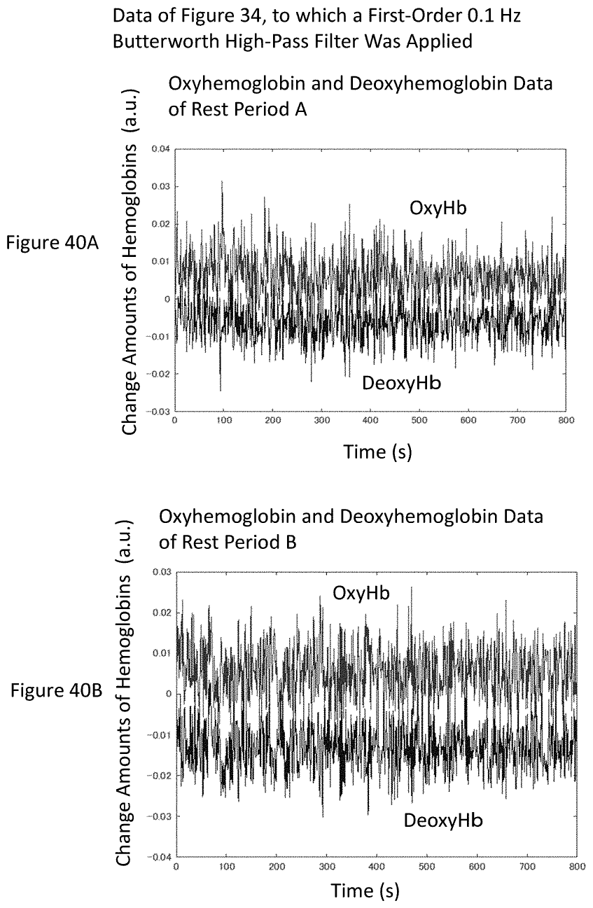

(5) Parameters concerning correlation characteristics of the axial directions of the 2 orthogonal axes on a 2-dimensional diagram (FIGS. 39-41).

Here, the “2 orthogonal axes” are a horizontal axis showing concentration change amounts of oxyhemoglobin (OxyHb axis) and a vertical axis showing concentration change amounts of deoxyhemoglobin (DeoxyHb axis), and a CBV axis and a COE axis created by rotating the above-mentioned horizontal axis and vertical axis by 45 degrees.

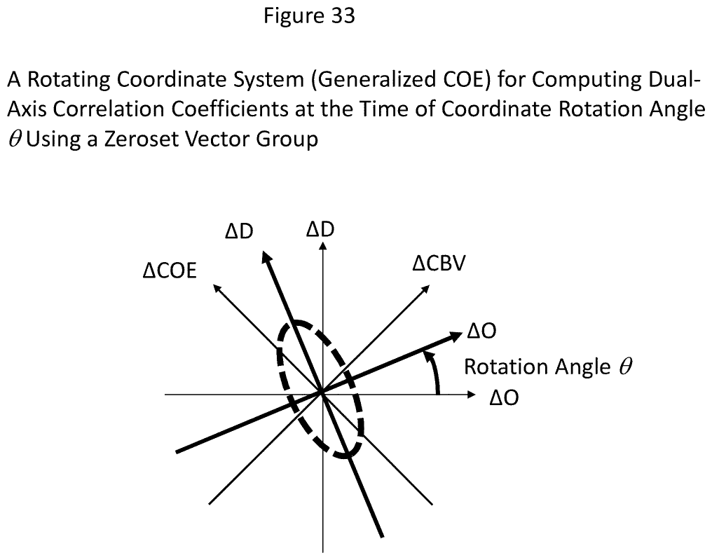

(6) Parameters using a vector group concerning a rotating coordinate system (FIG. 33).

(7) Parameters using a vector group concerning correlation coefficients of the two axial directions of the orthogonal axes on 2-dimensional diagrams with respect to the coordinate rotation angles on a rotating coordinate system (FIGS. 39-41).

(8) Parameters computed using derivatives of time course change amounts of oxyhemoglobin and time course change amounts of deoxyhemoglobin (FIG. 29A, FIG. 30A).

(9) Parameters computed using derivatives of time course change amounts of oxyhemoglobin and time course change amounts of deoxyhemoglobin, differentiated a plurality of times (here, twice) (FIGS. 29B, 30B).

(10) Parameters computed varying the sampling times (FIGS. 10A, 10B)).

(11) Parameters computed selecting unit width numbers that are sampling times multiplied by n (where n is an arbitrary number such that n>1) (FIGS. 10A, 10B).

Here, unit width numbers are displayed on the horizontal axis as 1=75 ms. By this means the numerical values of the units become concise, and graph display becomes easy to read.

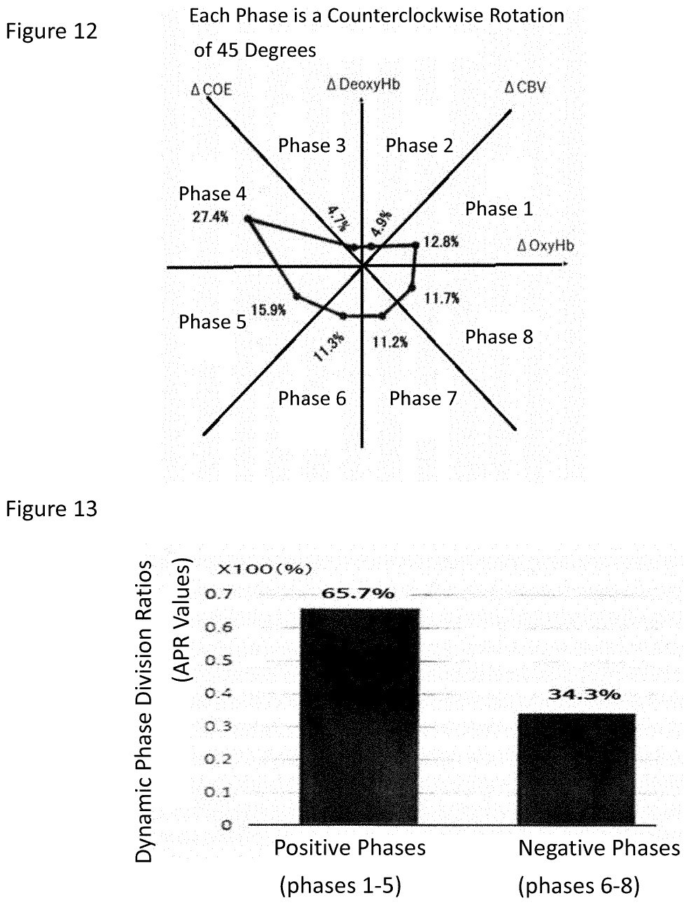

(12) Parameters, the values of which are data plotted on 2-dimensional diagrams, and displayed on display part 7 (FIG. 12).

Dynamic phase division ratios are displayed in FIG. 12, but they are not limited to these.

### Regarding Channels (ch)

In this specification, “channel (ch)” is used as a symbol representing a site of the brain of a living body (human), and the brain functions at the channels are as follows:

(1) Channels 1, 8 and 16 are sites of the right frontal lobe related to working memory concerning images.

(2) Channels 2, 3, 9, 10, 17, and 18 are sites in the right frontal lobe related to decision-making.

(3) Channels 7, 15 and 22 are sites of the left frontal lobe related to working memory concerning language

(4) Channels 5, 6, 13, 14, 20, and 21 are sites of the left frontal lobe related to judgment.

(5) Channels 4, 11, 12, and 19 are the anterior sites of the frontal lobe related to concentration.

### Background Art

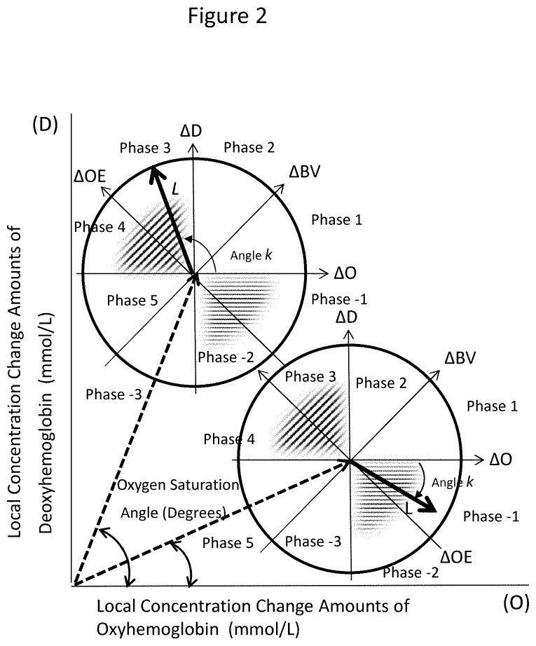

FIG. 2, in which the horizontal axis is concentration change amounts of oxyhemoglobin, and the vertical axis is concentration change amounts of deoxyhemoglobin, is an explanatory view that shows the vector model, showing the geometric relationship between the oxygen saturation angle and hemodynamic indices.

This explanatory diagram was presented at the international brain functional mapping conference OHBM2014 held in 2014, (The 20th Annual Meeting of the Organization for Human Brain Mapping; Jun. 8-12, 2014; Hamburg, Germany) by the present inventor (Toshinori Kato: A vector-based model of geometric relationships between oxygen saturation and hemodynamic indices).

Previously, functional measurements of the brain, muscles and the like were performed utilizing changes of oxyhemoglobin and deoxyhemoglobin.

Localized changes of oxyhemoglobin and deoxyhemoglobin are thought to give rise to changes in degree of oxygen saturation in the capillaries, due to tissue-capillary oxygen exchange. However, with functional measurements of NIRS, OIS (optical intrinsic signals), fMRI, etc., the relationship between changes in degree of oxygen saturation in the capillaries, and increases and decreases of oxyhemoglobin and deoxyhemoglobin remained almost completely unclarified. In fact, non-invasive brain functional mapping using changes in degree of oxygen saturation has not been reported.

Accordingly, a vector model of degree of oxygen saturation was constructed taking oxyhemoglobin and deoxyhemoglobin as vector components, making it possible to geometrically explain the relationship between changes in degree of oxygen saturation (ΔOS) and increases and decreases of ΔD, ΔO in the capillaries.

From this model, moving vectors on the O-D 2-dimensional plane can be defined as oxygen saturation change (ΔOS) vectors.

(1) The vector ΔOS is represented by a scalar L (amplitude), and a Phase k showing increases and decreases of ΔOS.

(2) ΔOS can be broken down by its polar coordinates into the 4 vector components of ΔD and ΔO, or ΔCOE (=ΔD-ΔO) and ACBV (=ΔD+ΔO).

(3) According to these 4 vector components, hemoglobin responses can be divided into 8 categories of 45 degrees each, out of 360 degrees.

(4) If degree of oxygen saturation at rest is 50%, the increase or decrease in ΔCOE and the change in ΔOS coincide.

(5) Independent of the value of the degree of oxygen saturation at rest, if ΔO decreases and ΔD increases, then ΔOS decreases, and if ΔO increases and ΔD decreases, then ΔOS increases.

(6) In cases of decreasing ΔO and decreasing ΔD, and increasing ΔO and increasing ΔD, the increase or decrease of ΔOS depends on the value of the degree of oxygen saturation at rest.

From FIG. 2, it can be seen that vector changes into phases 3 and 4, and, self-evidently, vector changes into the low oxidation phases of Phase −1 and Phase −2, are high oxidation [changes].

However, it was not clear on absolute coordinates of D and O where time at rest begins.

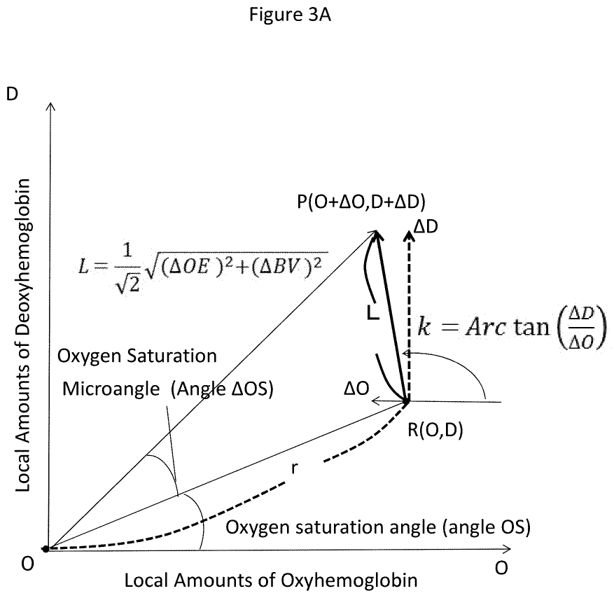

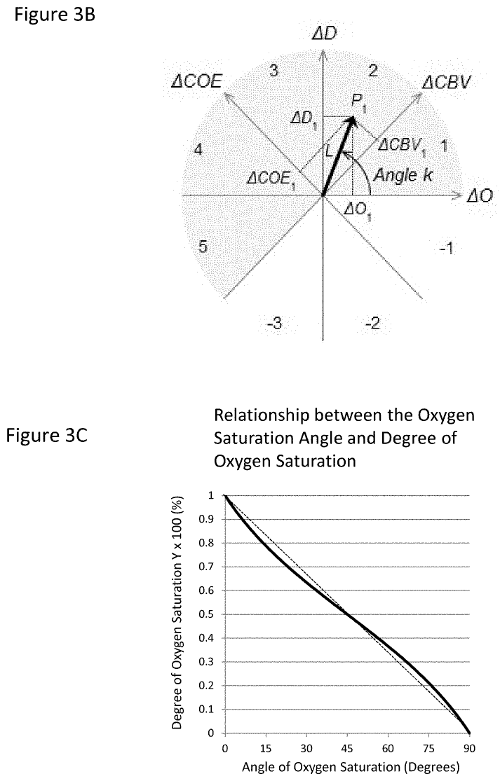

FIG. 3A is a graph for explaining the oxygen saturation angle and degree of oxygen saturation, in which the horizontal axis is concentration change amounts of oxyhemoglobin, and the vertical axis is concentration change amounts of deoxyhemoglobin.

In FIG. 3, the vectors moved on the O-D diagram are defined as oxygen saturation change vector[s] (ΔOS).

They are vector[s] transferred from R(O, D)to P(O+ΔO,D+ΔD), and ΔOS is represented by scalar L (amount moved) and phase K with respect to the O-axis (direction of movement).

Thus, “R” “P”=OP−OR=ΔOS (vector equation)

The angle between the 2 points is the angle of a triangle that takes L as its base, and it is defined as the angle ΔOS.

From (O, D) of the region of interest (ROI) measured, r and r1 are determined. This corresponds to the length of the base of the triangle.

When ΔOS moves in a clockwise direction, the degree of oxygen saturation increases. When ΔOS moves in a counter-clockwise direction, the degree of oxygen saturation decreases. In fact, it is possible to determine the actual value of OS by a diagram of the relationship between oxygen saturation and the angle OS.

1 FIG. 3B is a graph showing phase divisions, in which the horizontal axis is concentration change amounts of oxyhemoglobin, and the vertical axis is concentration change amounts of deoxyhemoglobin.

On a vector polar coordinate plane used for vector analysis, when a vector joining the origin to arbitrary point P₁(Δ O₁, ΔD₁) is transformed to the ΔCOE/ΔCBV axes, the coordinates of ΔCOE₁ and ΔCBV₁ can be obtained.

The numbers on arcs of the circle show the phase numbers. Among the 8 octants divided by the 4 axes, the phases showing increasing ΔD or increasing ΔCOE (gray areas) show low oxygenation or deoxygenation, and are phases showing rising brain activity.

On the other hand, in octants showing decreasing ΔD and ΔCOE (white areas), there is almost no rise in brain activity.

The index that quantitatively shows these oxygen metabolism phases is the angle k.

FIG. 3C is a graph in which the horizontal axis is angle of oxygen saturation (degrees) and the vertical axis is degree of oxygen saturation, showing the relationship between the oxygen saturation angle and the degree of oxygen saturation.

Y=O/(O+D)=1/[1+tan(angle OS)]

The relationship between oxygen saturation and angle OS is given by the graphs of FIG. 3, and in fact determines the value of OS.

When ΔOS moves in a clockwise direction, the degree of oxygen saturation increases.

When ΔOS moves in a counter-clockwise direction, the degree of oxygen saturation decreases.

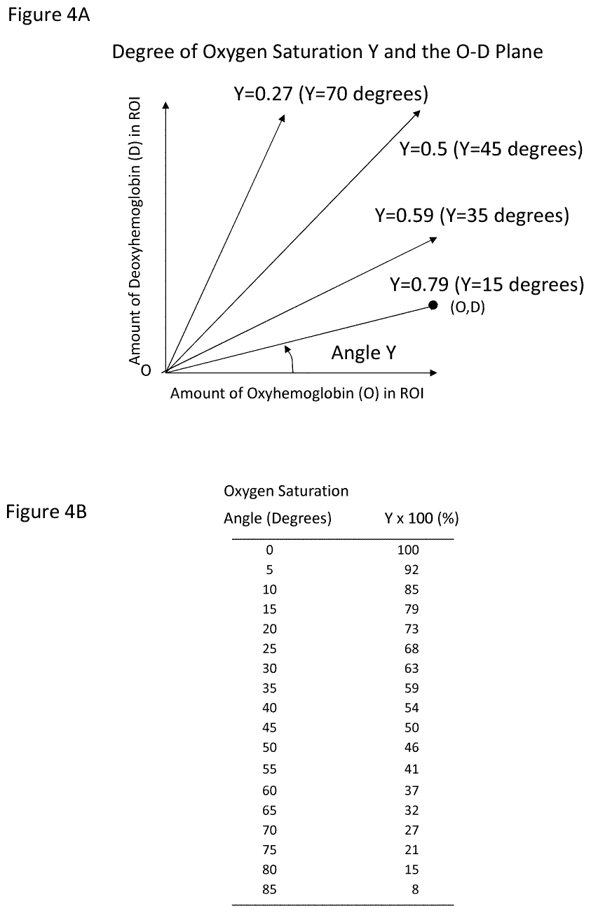

FIG. 4A is a graph in which the horizontal axis is the concentration change amount of oxyhemoglobin, and the horizontal axis is the concentration change amount of deoxyhemoglobin, showing the relationship between the degree of oxygen saturation and the angle Y; and FIG. 4B is a table corresponding to those.

When the slope on the O-D plane is taken as angle Y, then:

Oxygen saturation Y=1/(1-arctan (Y))

In the region of interest (ROI),

Blood volume BV (θ)=O(t)+D(t)

### Method For Evaluating Biological Function

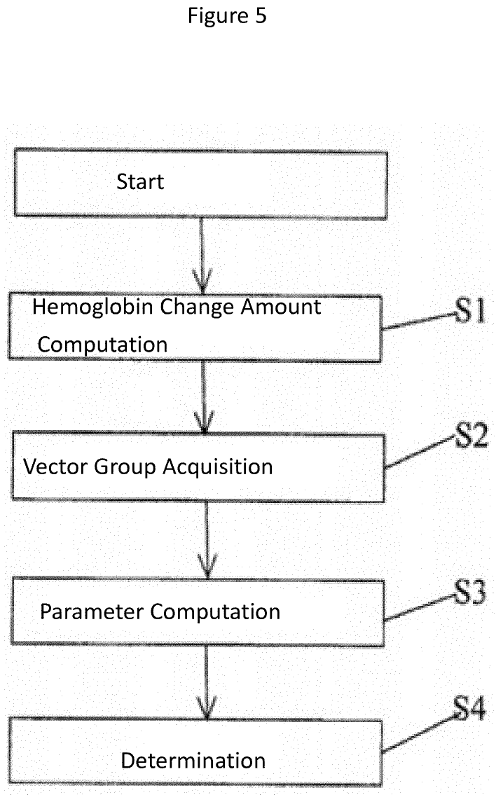

FIG. 5 is a flow chart for explaining the operation of the apparatus for evaluating biological function of an embodiment of the present invention.

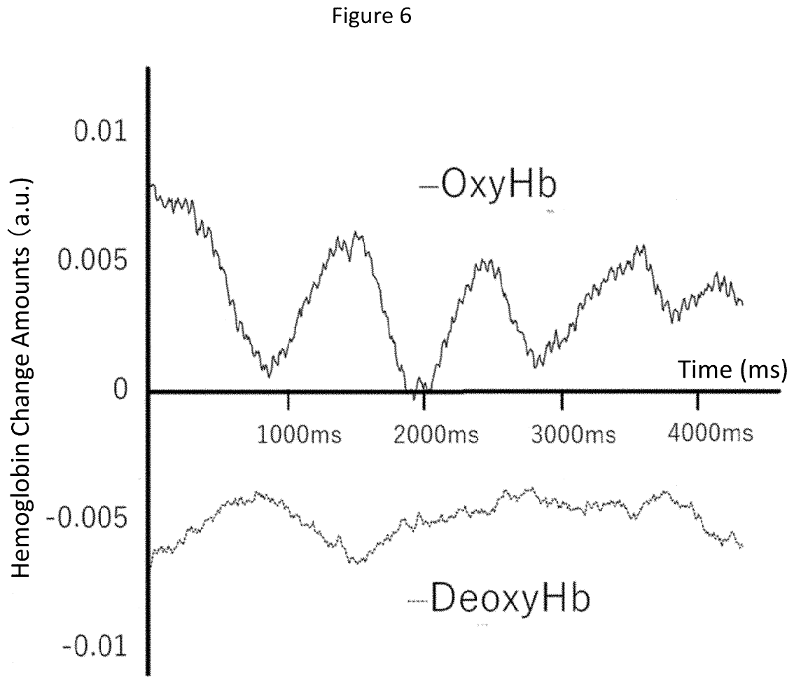

FIG. 6 is a figure showing the procedure for computing the dynamic phase division ratio; it is a graph of acquired data in which the horizontal axis is time, and the vertical axis is change amounts of hemoglobin.

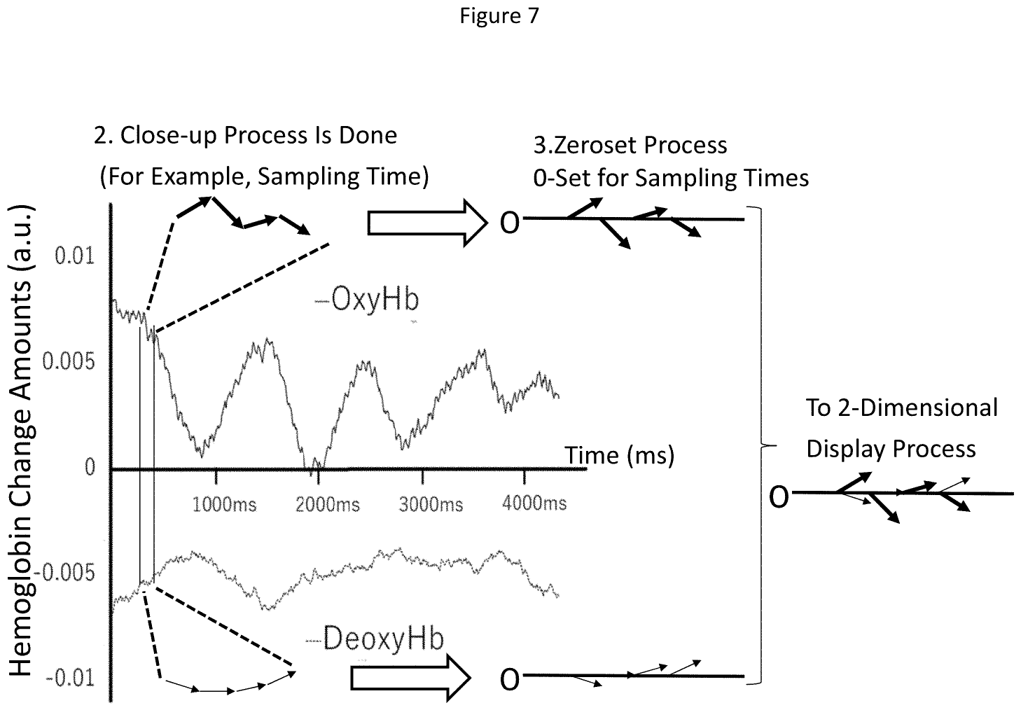

FIG. 7 is a graph in which the horizontal axis is time and the vertical axis is change amounts of hemoglobin; it shows the procedure whereby an arbitrary interval is selected, unit widths are set, and zeroset processing is done.

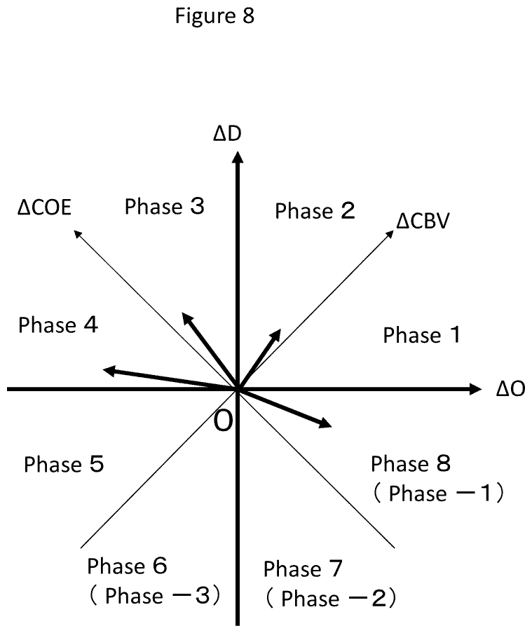

FIG. 8 is a graph in which the horizontal axis is concentration change amounts of oxyhemoglobin and the vertical axis is concentration change amounts of deoxyhemoglobin, showing the procedure whereby the group of zeroset vectors are classified into phase divisions.

First, as shown in FIG. 6, time course change amounts of oxyhemoglobin and time course change amounts of deoxyhemoglobin are computed, based on light information from the detection parts (Step S1).

Next, as shown in FIG. 7, based on 2-dimensional diagrams showing the relationship between change amounts of oxyhemoglobin and change amounts of deoxyhemoglobin, a group of zeroset vectors is obtained, for each prescribed sampling time (for example, 75 ms) (Step S2).

The zeroset values of oxyhemoglobin and deoxyhemoglobin are considered to be vector components (ΔO, ΔD), and phase angles k and scalars L are computed (zeroset vectors), and plotted on a graph on a 2-dimensional diagram (Step S3).

Subsequently, as shown in FIG. 8, dynamic phase division ratios (APR values) are computed, showing how frequently the vector group appear in phase divisions 1-8 of the plurality of divisions (here, 8), based on 2-dimensional diagrams (Step S4).

The 8 phase divisions shown in FIG. 8 are divided by orthogonal axes: a horizontal axis showing concentration change amounts of oxyhemoglobin (OxyHb axis) and a vertical axis showing concentration change amounts of deoxyhemoglobin (DeoxyHb axis); and by a CVB axis and a COE axis, formed by rotating the above-mentioned horizontal axis and vertical axis by 45 degrees.

For example, a case in which the frequency of appearance of each phase and the resulting ratios are as below:

Phase 1=0 0/4 (0%)

Phase 2=1 1/4 (25%)

Phase 3=1 1/4 (25%)

Phase 4=1 1/4 (25%)

Phase 5=0 0/4 (0%)

Phase 6=0 0/4 (0%)

Phase 7=0 0/4 (0%)

Phase 8=1 1/4 (25%)

Thus, because 4 zeroset vectors were obtained in an arbitrary interval, if, for example, phases 1-5 are taken as active phases, the dynamic phase division ratio (APR value [active phase ratio])=3/4 (75%).

After that, the state of biological function is determined based on the computed dynamic phase division ratios (Step S4).

Here, on the polar coordinate plane comprising 4 axes shown in FIG. 8, vectors are displayed, having as their components the 4 types of hemoglobin indices of ΔO (Aoxyhemoglobin), ΔD (Adeoxyhemoglobin), ΔCBV and ΔCOE.

By means of the combinations of increase and decrease of the 4 vector components, the 8 octants on the vector plane can be divided as 8 phases.

Namely:

Phase 1: 0<ΔD and ΔCOE<0

Phase 2: 0<ΔO and 0<ΔCOE

Phase 3: ΔO<0 and 0<ΔCBV

Phase 4: 0<ΔD and ΔCBV<0

Phase 5: ΔD<0 and 0<ΔCOE

Phase 6: ΔD<0 and 0<ΔCBV

Phase 7: 0<ΔO and ΔCBV<0

Phase 8: ΔO<0 and ΔCOE<0

The vectors can be classified as shown above. The 8 phases are related to changes in degree of tissue oxygen saturation of the measurement sites, shown below. This is also clear from O-D diagrams consisting of amounts of oxyhemoglobin (O) and amounts of deoxyhemoglobin (D) in the tissues.

From the frequency of occurrence of phases, detected from the measured intervals of time, distance, or the like, it is possible to judge the following.

Along the ΔCBV axis, when the frequency of occurrence of phases 1, 2, 3, and 8 increases over 50%, it shows an increase of blood volume of the measured site, and is judged to be a hyperemic state. When the frequency of occurrence of phases 4, 5, 6, and 7 increases over 50%, it shows a decrease in blood volume of the measured site, and is judged to be an ischemic state.

In cases when 50% is sustained, it is judged that there is hardly any change in blood volume.

Frequency of occurrence of phases 1 through 5, which are the phases of ΔCOE increase or ΔD increase, shows increased tissue oxygen consumption from at rest.

On the other hand, when phases 6, 7, or 8 increase, this shows that the blood supply has increased.

Furthermore, as shown in the “vector model showing the geometric relationship between oxygen saturation and the OS angle”, when the frequency of occurrence of phase 3 and phase 4 increases regardless of the value of tissue oxygen saturation at rest, a very strong drop in tissue oxygen saturation and a low oxygenation response are diagnosed, and it can be determined that the brain has consumed oxygen very actively.

Contrarily, when phase 6 and phase 7 increase, rising tissue oxygen saturation and a strong high oxygenation response are diagnosed, and it can be determined that fresh oxygen has been supplied to the brain.

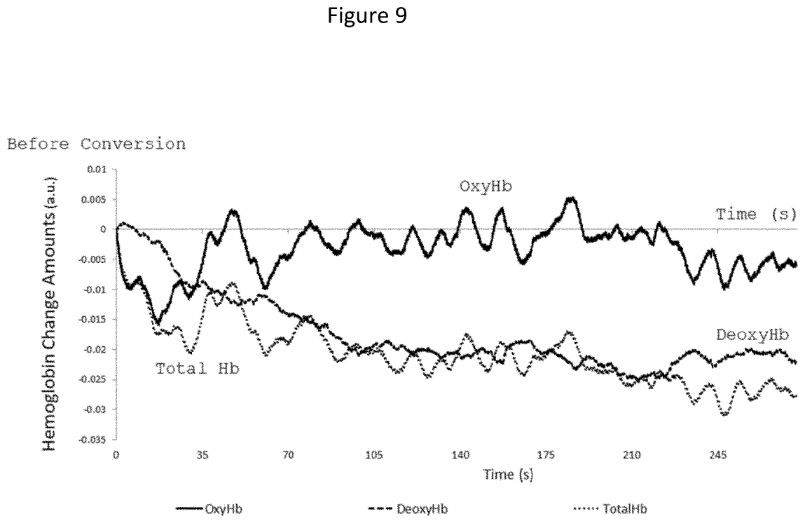

FIG. 9 is a graph before conversion, showing a computation example of the dynamic phase division ratio, in which the horizontal axis is time, and the vertical axis is change amounts of hemoglobin (oxyhemoglobin, deoxyhemoglobin, and total hemoglobin).

As for data processing performed using an orthogonal vector plane formed by a ΔOxyHb vector (ΔO) and a ΔDeoxyHb vector (ΔD), ΔO and ΔD at each arbitrary unit of time (milliseconds) were offset and taken as vector origins.

In order to prevent distortion of the task starting point of ΔO and ΔD from high-frequency noise and compute change amounts during the task as accurately as possible, there are cases when a 0.1 Hz low-pass filter process (Butterworth filter) is implemented on the raw ΔO and ΔD data.

The purpose of this process was “smoothing”, reducing errors in vector origins computed at very small intervals. From ΔO and ΔD data that underwent this process, the vector components (the 4 indices ΔO, ΔD, ΔCOE, and ΔCBV) are computed by means of Equations 1 and 2.

By means of the ratio of ΔCOE to ΔCBV, obtained from equations 1 and 2, (or the ratio of ΔD changes to ΔO), [the ratio? this is not clear to me] degree of oxygen exchange: angle k (Equation 3) is defined as a quantitative index of the strength of oxygen metabolism.

The ratio K is shown in (Equation 4).

### Mathematical Expression 1

Zeroset vector (k, [symbol] L)

[symbol]

COE=(ΔD−ΔO)/√2 (Equation 1)

ΔCBV=(ΔD+ΔO)/√2 (Equation 2)

“k”=can be determined by Equation(3), as shown below, using the angle k between the vector and the positive ΔO axis.

$$\begin{matrix}k = Arc tan ( \frac{Δ D}{Δ O} ) = Arc tan ( \frac{Δ C B V}{Δ C O E} ) + 4 {5}^{∘} ( - 13 {5}^{∘} ≦ k ≦ 22 {5}^{∘} ) & ( Equation 3 ) \\ Ratio K = Δ O / Δ D & ( Equation 4 ) \\ {( Δ L )}^{2} = {( Δ O )}^{2} + {( Δ D )}^{2} & ( Equation 5 )\end{matrix}$$

The active phase ratios (APRs) of the region of interest (ROI) are determined by Equation 6. The number of trials in which k increases, out of the total number of trials, is defined as the dynamic phase division ratio, equal to APR (active phase ratio, %), and was computed for each channel measured.

$$Mathmatical expression 2$$

$$APR ( \% ) = \frac{\text{Number of trials shown in an active phase (n)}}{\text{Total number of trailas (n)}} \times 100 \\ APR denominator = ( \text{prescribed time, or distance} ) \times ( \text{number of people} ) \times ( \text{number of sites measured} ) \\ APR numerator = number of active phase trials among the total number of trials ( used as the denominator ) \qquad ( Equation 6 )$$

In the numerator, “trials” may be divided into male or female, adults or children, or by age, to learn special characteristics with respect to a population; and even among individuals, by measuring a plurality of times, the average probability of active phases can be computed.

In addition, arbitrary active phase ratios can be computed. Even at rest, by selecting arbitrary phases, we found that they show constant values.

In addition, in the case of individuals, it becomes possible to group sites together to quantify the state at rest of the right brain and the left brain.

FIG. 10A is data in a resting state of a period of 224 seconds; and when phases 1-5 are defined as active phases (APR+), they show an almost constant trending of 0.7-0.8 from unit widths of 75 ms to 7.5 seconds (75 ms×100). Because fundamental vibration data is thought to be abundantly included in the APR itself, it was assumed that if unit widths were changed, results would change in a fairly big way, but looking at changes in the positive (+) phases and the negative (−) phases, we determined that there was not that much change and almost no dependence on unit widths, and that this reflected the fact that fluctuations of the fundamental vibrations are random.

On the other hand, individual APR phases are changed greatly by unit widths. From FIG. 10B, it can be seen that phase divisions 1, 4 and 5 vary easily depending on the unit widths. These changes reflect the angular distribution of the fundamental vibrations.

FIG. 11 is a graph for confirming the dependence on unit width, taking phases 2, 3, 4, 5 and 6 as active phases and phases 1, 7 and 8 as inactive phases; it is a graph showing the horizontal axis as unit width numbers (1=75 ms) and the vertical axis as dynamic phase division ratio (APR values) for confirming dependence on unit width.

As shown in FIG. 11, the dynamic phase division ratios (APR values) depend greatly on unit width, and taken together with FIG. 10A, the COE axis is shown to be a cause of the large dependence on unit width. This is also in accord with the fact that APR angular distribution is high in the direction of the COE axis. This is one reason that it could be a more valid index, compared to Aoxyhemoglobin, Adeoxyhemoglobin, and the like, and it shows that the state at rest tends to move on the COE axis.

Consequently, by obtaining an angular index that erases unit width dependence, quantification at rest is possible.

Phases should be defined so that the phases are selected to maximize angular frequency of occurrence. Accordingly, dual axis correlation coefficients of a rotating coordinate system are computed to define the angle for which the angle frequency of occurrence is greatest.

FIG. 12 is a graph in which time course data are plotted on a 2-dimensional diagram where the horizontal axis is concentration change amounts of oxyhemoglobin and the vertical axis is concentration change amounts of deoxyhemoglobin; it is divided into 8 phase divisions and displayed in radar chart format on the display part; and the dynamic phase division ratio, which is the proportion (%) of each phase, is displayed on the display part. Because on the radar chart the data are plotted on the coordinate axes of Δdeoxyhemoglobin, Δoxyhemoglobin, ΔCOE, and ΔCBV, and because it is thus possible to determine at a glance, from the shape of the octagon, the coordinate on which it depends and whether the shape is concave or convex, it is thus possible to distinguish a plurality of resting state patterns to evaluate them.

FIG. 12 is the analysis results of data obtained from the region corresponding to Brodmann's areas 44 and 45 of the right frontal region the head of a 21-year-old male, a healthy university student. (Subject was measured in 2017 with the subject seated in a chair in front of an fNIRS apparatus in a resting state, with eyes closed.)

1600 zero[set] vectors, were created from 120 seconds of data, every 75 ms; results of analysis. The phases and frequency of occurrence of 1600 zero set vectors become a table, and when displayed by percents, a graph.

Here, as APR computation example 1:

Phase 1: 12.8%

Phase 2: 4.9%

Phase 3: 4.7%

Phase 4: 27.4%

Phase 5: 15.9%

Phase 6: 11.3%

Phase 7: 11.2%

Phase 8: 11.7%

In addition, frequency of occurrence of the phases are:

Phase 1: 205

Phase 2: 79

Phase 3: 75

Phase 4: 439

Phase 5: 255

Phase 6: 181

Phase 7: 179

Phase 8: 187

Now, when the vectors of a vector group are classified into the phases, if a vector falls on an axis, it can be divided between 2 phases with a frequency of occurrence of 0.5 in each. Or, they may be counted in the phase on the clockwise rotation side, or counted on the counterclockwise side.

FIG. 13 is a bar graph showing dynamic phase division ratios (APR values) in plus (+) phases (phases 1-5) and minus (−) phases (phases 6-8). Phases 1-5, in which Adeoxyhemoglobin or ΔCOE are increasing, are defined as plus phases, in which the brain is active (active phases), and phases 6-8 are defined as minus phases (inactive phases).

Dynamic phase division ratios (APR values) of an arbitrary time and arbitrary channels were able to be quantified as 65.7%.

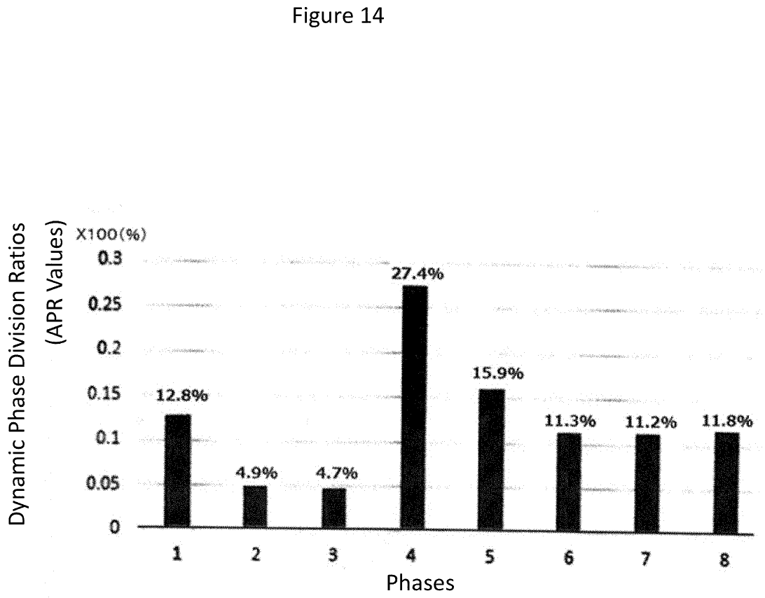

FIG. 14 is a bar graph in which the horizontal axis is phases, and the vertical axis is dynamic phase division ratios (APR values).

It can be seen from FIG. 14 that the dynamic phase division ratio (APR value) of phase 4 is high, and the dynamic phase division ratios (APR values) of phase 2 and phase 3 (among others) are low.

The relative frequency of phases 4 and 5 taken together is high, at 43.3%, and the total for phases 2 and 3 is low, at 9.6%; from the fact that the ratio between the two groups of phases shows as 9:2, it can be determined that an ischemic state is contributing even more than a ΔCBV increase to low oxygenation, in which ΔCOE increases.

From the fact that the total for the inactive phases 6, 7, and 8 is 34.2%, and the active phase total is 65.8%, it can be determined that the subject, although in a resting state with his eyes closed, is having heightened activity of the right frontal region of his head.

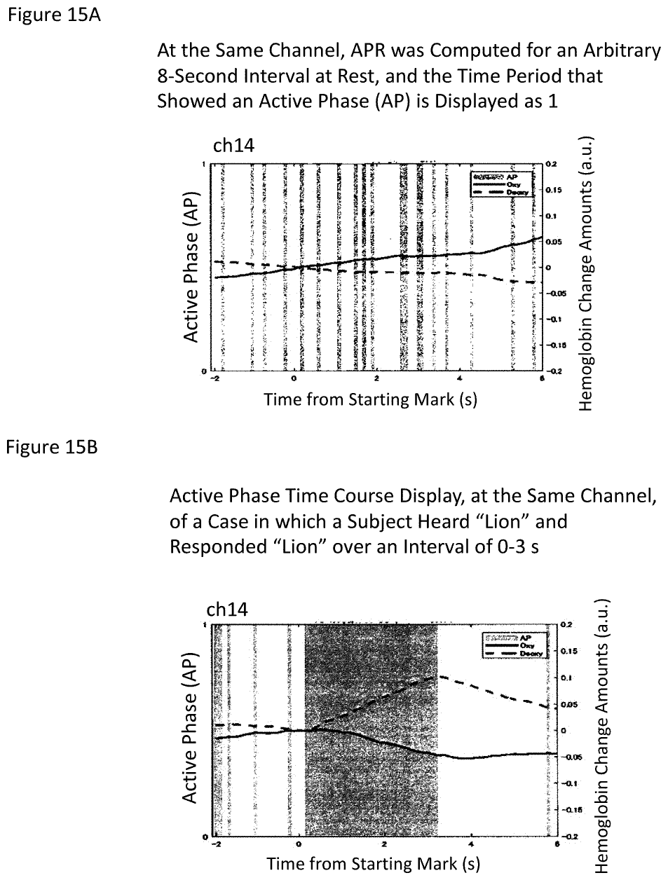

In FIG. 15A, computations are done for an arbitrary 8 s period at rest at the same channel (ch14); and in 15B, at the same channel (ch14), during a period from 0 s to 3 s, a task of hearing the word “lion” and responding “lion” is repeated 8 times; and for that time, at the measurement site, for a total of 8 s, from 2 s before “lion” is said until the subject finishes speaking, average oxyhemoglobin change amounts, average deoxyhemoglobin change amounts, and the time periods showing active phases (AP) are computed, compared and displayed in time course series.

From FIG. 15A, at ch14, sporadic increases in active phases are occurring at rest. The motor speech area corresponding to ch14 works not only in external speech (vocalized) but also in inner speech (not vocalized, as in the case of reading to oneself or thinking in words). For that reason, it can be determined that even at rest, an increase in active phases is occurring in cases when the subject did language activity.

In the graph in FIG. 15B, there is “Time from the Mark”, but the starting point mark is the instant “lion” was begun to be spoken, speech was finished in approximately 600 ms, and 300-400 ms after speech was finished, the subject begins to say “lion”. Namely, from the subject's beginning to hear “lion” even until she/he begins to speak, the period of time showing active phases is increasing continuously.

While FIGS. 15 A and B are at the same channel, increases in dynamic phase division ratios (APR values) are occurring sporadically at rest, and during the speech task trials, they coincide with deoxyhemoglobin increase intervals, and thus it can be determined that increases of dynamic phase division ratios (APR values) are persisting.

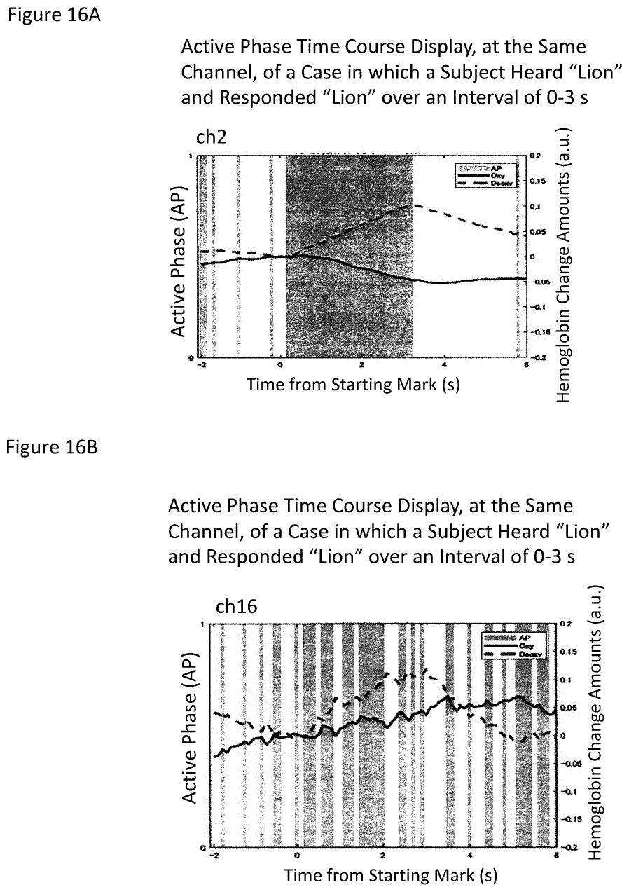

In FIG. 16A, computations are done for an arbitrary 8 s period at rest at the same channel (ch2); and in 16B, at the same channel (ch16), during a period from 0 s to 3 s, a task of hearing the word “lion” and responding “lion” is repeated 8 times; and for that time, at the measurement site, for a total of 8 s, from 2 s before “lion” is said until the subject finishes speaking, average oxyhemoglobin change amounts, average deoxyhemoglobin change amounts, and the time periods showing active phases (AP) are computed, compared and displayed as time course series.

From FIG. 15 and FIG. 16, at ch14 and ch2/ch16, at rest and during task load, the graphs differ. Ch14 is the motor speech area, and a strong increase in dynamic phase division ratio (APR values) can be perceived. Ch16 is a language area but it is apart from the region for speech, and so its continuity is interrupted.

Ch2 is in the right side of the brain, and responds after ch14, and [its] dynamic phase division ratios (APR values) are increasing even after speech is finished; thus even though they are contralateral in the right brain and left brain, it can be seen that they are acting with different functions. Ch2 is a right brain site, and located contralateral to ch14. There are also cases when it responds accompanying language, but it is active when one is about to communicate visual images. It was selected for comparison with ch14.

In different channels, during language task trials, in cases when increasing APR values coincide with intervals of increasing deoxyhemoglobin, it can be determined that the dynamic phase division ratios (APR values) are not necessarily increasing continuously in the intervals of deoxyhemoglobin increase intervals.

Now, ch14 is a site of the left brain related to the language area related to conversation. Because it is considered to be a site essential for answering “lion”, it is easily compared with time at rest.

It can be seen that Ch14 does not return to [its state] at rest during the time measured.

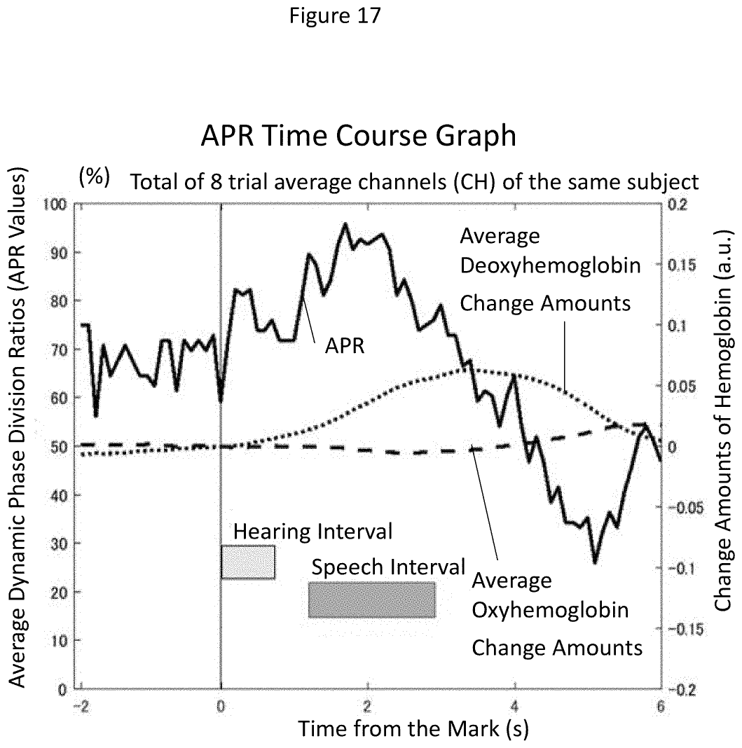

FIG. 17 is a graph in which, at the same channel, for 8 trials at ch15 in a case in which during the period from 0 s to 3 s a subject hears the word “lion” and answers “lion”; and for a total of 8 s, including 2 s before the hearing interval and 3 s after the speaking intervals, average oxyhemoglobin change amounts, average deoxyhemoglobin change amounts, and average dynamic phase division ratios (average APR values) (%) are computed, compared, and displayed as time course series.

ch15 is adjacent to ch14 and corresponds to a language field related to conversation in the left brain like ch14. ch16 is adjacent to ch15 and in the vicinity of the language area related to conversation.

As shown in FIG. 17, in the 2-second interval at rest, the dynamic phase division ratios (APR values) trended around 60-70%, increased with the task, and at around 2 s exceeded 90%.

In addition, 4 seconds after task start, they showed 50%, 5 seconds after, decreased to 30%, and 6 seconds after, returned to 50%.

Due to the task, from time at rest, APR values increased by approximately 20%. A phenomenon of an approximate decrease of 30% is detected.

Namely, even in the interval of hearing “lion”, APRs are increasing, and in the speaking interval, the APR increase is peaking before speech is completed. Furthermore, in the recovery period, it can be seen that the APR shows even lower values than at rest. On the other hand, because average deoxyhemoglobin change amounts show peak values after speaking is finished, the processes tracked by APR values cannot be explained even by average deoxyhemoglobin change amounts.

Time course changes of this kind of the dynamic phase division ratios (APR values) cannot be predicted at all by conventional quantitative displays of average oxyhemoglobin change amounts and average deoxyhemoglobin change amounts. This supports the fact that the dynamic phase division ratio (APR values) is a new quantitative index of brain activity.

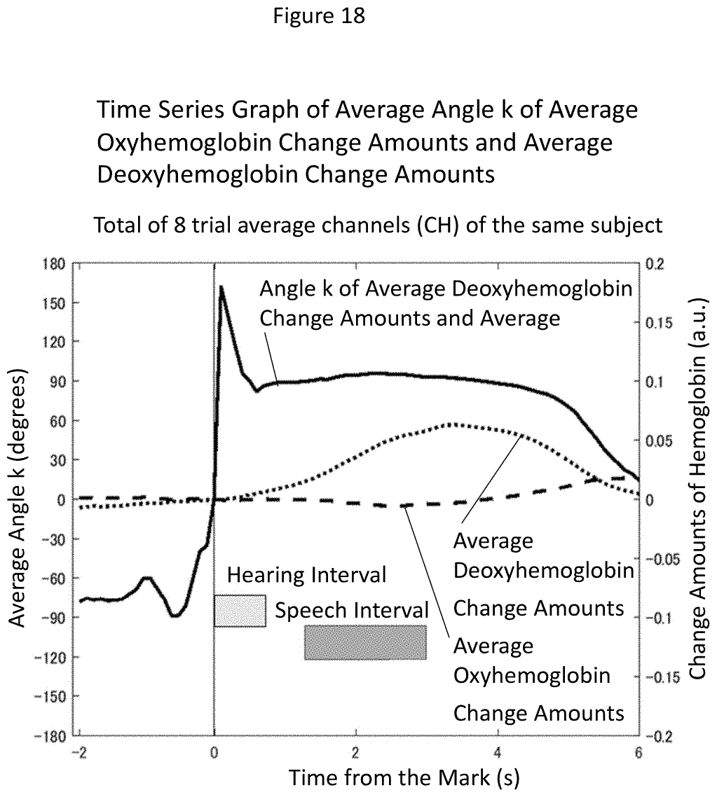

FIG. 18 is a graph in which, at the same channel, for 8 trials at ch15 in a case in which during the period from 0 s to 3 s [a subject] hears the word “lion” and answers “lion”; and for a total of 8 s, including 2 s before the hearing interval and 3 s after the speaking intervals, average oxyhemoglobin change amounts, average deoxyhemoglobin change amounts, and average angles k (degrees) are computed, compared, and displayed as time course series.

Average angles k (degrees) peak in the hearing interval, and without decreasing even when the speaking interval is finished, during the task and completion of the task cannot be detected.

Time course changes of this kind of dynamic phase division ratios (APR values) cannot be predicted at all by conventional angle k displays. It is not possible to differentiate between time at rest and during a task period by the angle k. Comparing FIG. 17 and FIG. 18 supports the fact that the dynamic phase division ratio (APR values) is a new quantitative index for brain activity, not only for time at rest.

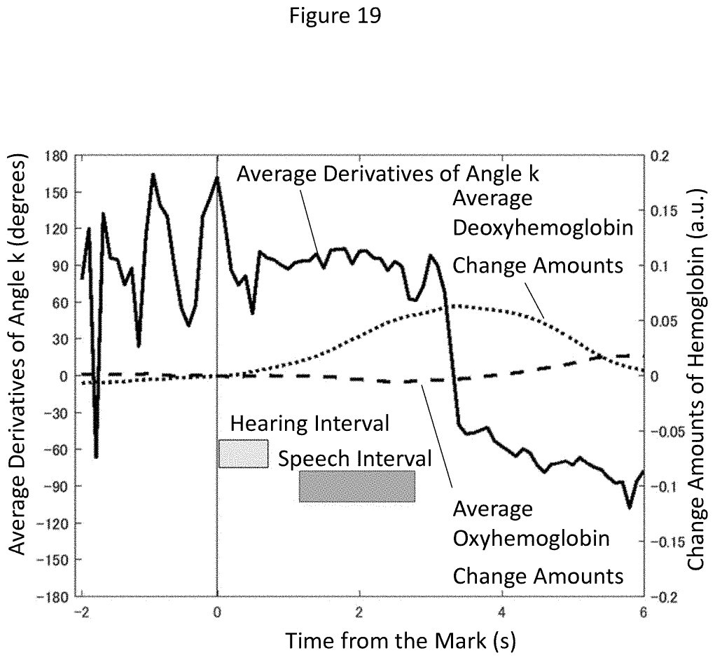

FIG. 19 is a graph in which, at the same channel, for 8 trials at ch15 in a case in which during the period from 0 s to 3 s a subject hears the word “lion” and answers “lion”; and for a total of 8 s, including 2 s before the hearing interval and 3 s after the speaking intervals, Δaverage oxyhemoglobin change amounts, Δaverage deoxyhemoglobin change amounts, and angle k differentials, computed from differentials of average change amounts of oxyhemoglobin and differentials of Δaverage change amounts of Δdeoxyhemoglobin, were computed, compared, and displayed as time course series.

As shown in FIG. 19, in the differential angle k display, variations in the angle are greater during rest than during the task, and it is difficult to distinguish between time at rest and during the task period. The peak of the speech segment, which was able to be detected by the APR, cannot be observed.

Completion of the speech segment is easy to see, but it is an index in which the implications of plus and minus angles are difficult to understand.

Namely, from FIG. 17, FIG. 18, and FIG. 19, it can be seen that by means of dynamic phase division ratios (APR values), measurements that could not be detected by computing angle k, differential angle k, average oxyhemoglobin change amounts and average deoxyhemoglobin change amounts are possible.

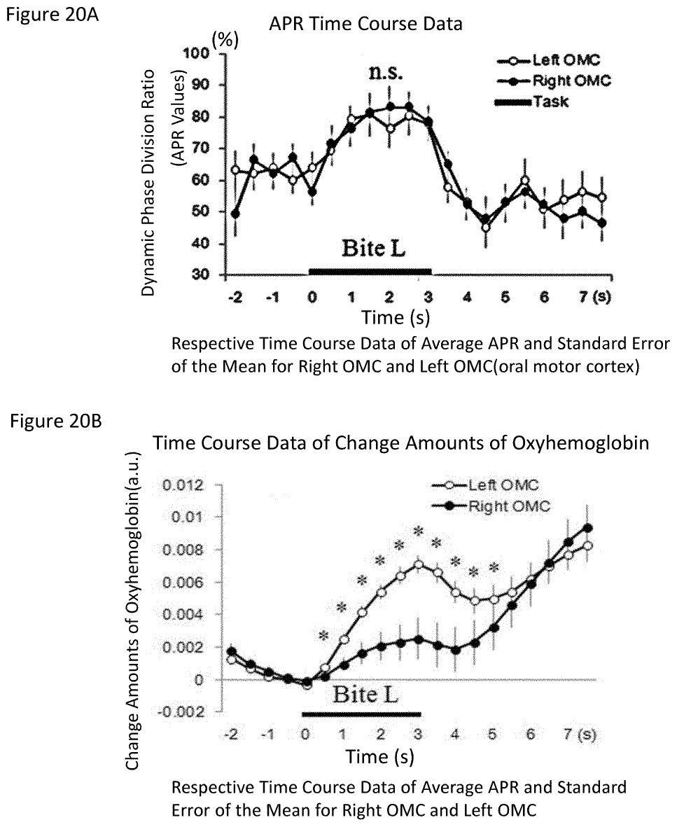

FIG. 20 is graphs in which, in 20A, the horizontal axis is time (s) and the vertical axis is dynamic phase division ratios (APR values) (%); and in 20B, the horizontal axis is time (s) and the vertical axis is change amounts of oxyhemoglobin; 46 channels were disposed on the top of the head, a task was performed in which a subject strongly bit down on an object for 3 s with the left masseter muscle, the results were compared with time at rest, and time course data of change amounts of oxyhemoglobin and active phase ratio values (APR) in that time are compared.

Asterisks show the intervals of significant differences (z>2.0) between the right oral motor area (oral motor cortex: OMC) and the left oral motor area (oral motor cortex: OMC). The time course data of change amounts of oxyhemoglobin is rising also after the task, but for the APR values, differences were significant only during the task. The significance only during the task is because in a task of moving the masseter muscle, the OMC is activated in order to pull up the lower jaw, competing against the force of gravity, and at rest, because the masseter muscle is unused, and relaxes, following the law of gravity, OMC activity returns to rest.

FIG. 21 is mapping figures for which a task was performed in which a plurality of channels were disposed on the top of the head, and [the subject] strongly bit down on an object for 3 s with the left masseter muscle, and the results were compared with time at rest; 21A is APR mapping of the left side masseter muscle task, and 21B maps change amounts of oxyhemoglobin in the left side masseter muscle task.

Asterisks show high frequency active sites (z>2.0). ROI sites, surrounded by dotted lines, are the oral motor area (sites that move the mouth and oral cavity, OMC), identified by MRI, and sites outside the ROI dotted lines are not in the OMC. If the APR as an indicator of brain activity index is valid, then inside the ROI, APR increases can be expected to occur during the task. Ch40 of the right ROI, and ch1 and ch2 of the left ROI, were shown to be statistically significant.

Because it is known that whether right biting or left biting, they are regulated from both the left brain and the right brain, OMC activity was anticipated in both the left brain and right brain; but the oxyhemoglobin change amount mapping, sites outside the OMC were also activated in the left brain. However, APR mapping was able to detect OMC activity of both the left brain and the right brain. Mapping using APR values can be seen to be a more highly sensitive index than the conventional index of oxyhemoglobin. APR values are quantitative mapping; conventional indices are qualitative mapping.

Namely, in cases when oxyhemoglobin is the index, the time up to just before the task start must be set as time at rest, and because this is qualitative, even if a statistical comparison between rest time and task time was done, the meaning of the difference between the two states can only be conjectured. The size of the difference between the two states is also relative. If APR, as for example, a difference of 10%, comparison with other research data is possible, and in the end it is only about whether there is a statistical difference, and it becomes a question of comparison with other research data. By the use of zeroset vectors at rest, in the present example, it is easily seen from the percentage display whether the same state as that at rest is interrupted; but with only oxyhemoglobin values, perhaps deoxyhemoglobin values might be indicators of change, but by only oxyhemoglobin values not only is it impossible to determine rest, but errors of judgment are also possible.

In fact, after the task is completed, even though the masseter muscle should not be working, oxyhemoglobin is still increasing.

With oxyhemoglobin, there is a possibility of misdiagnosis of the state after the task. With APR, after the task, it can be seen to be returning to the numerical values of the resting state. In this way, APR makes it possible to accurately evaluate the recovery process.

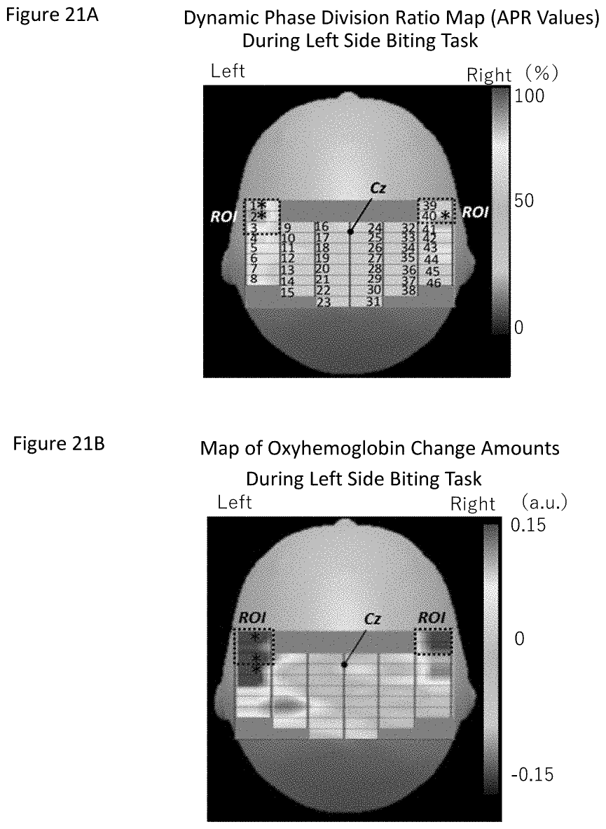

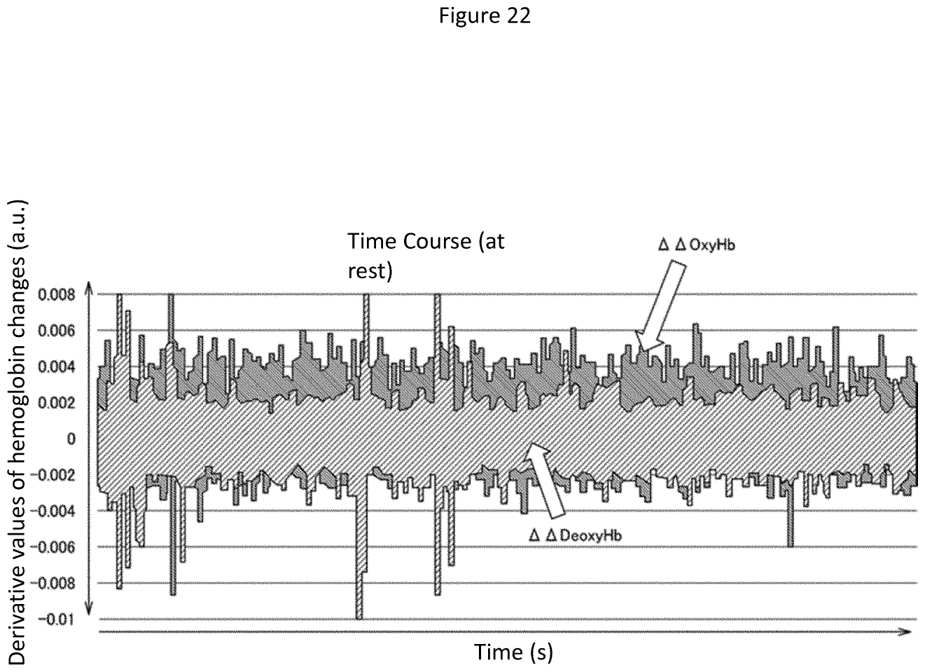

FIG. 22 is a time course graph in which the subject's measured data at rest shown in FIG. 9 are each differentiated (ΔΔOxyHb, ΔΔDeoxyHb); the horizontal axis is time and the vertical axis is derivatives of concentration change amounts of oxyhemoglobin (ΔΔOxyHb) and derivatives of concentration change amounts of deoxyhemoglobin (ΔΔDeoxyHb), to show their relative changes.

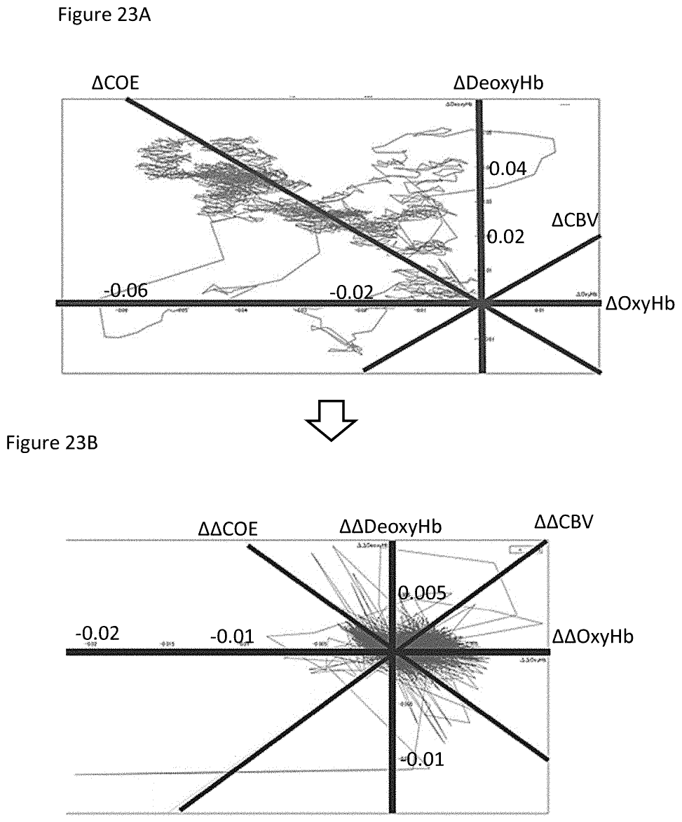

FIG. 23A is a 2-dimensional diagram in which the subject's measured data at rest shown in FIG. 9 (ΔOxyHb, ΔDeoxyHb) are displayed respectively as the horizontal axis and the vertical axis; 23B is a 2-dimensional diagram in which the data of FIG. 22 (ΔΔOxyHb, ΔΔDeoxyHb) are displayed respectively as the horizontal axis and the vertical axis.

From FIG. 23A, the data can be determined to be oscillating at a location near the COE axis, not the CBV axis.

From FIG. 23B, it can be seen that oscillation of ΔO is greater than that of ΔD, and is consolidated at the origin (0) of the graph.

FIG. 24 is an explanatory view showing a case in which the phase diagram is divided into 24 divisions. For 8 divisions, it is divided so that one phase is 45 degrees, but for 24 divisions, each one phase of the 8 divisions is further divided into 3 equal parts of 15 degrees each.

For 8 divisions, division 1 corresponds to phase 1, 2 to phase 2, 3 to phase 3, 4 to phase 4, 5 to phase 5, 6 to phase −3, 7 to phase −2, and 8 to phase −1. For 24 divisions, divisions 1, 2 and 3 correspond to phase 1; 4, 5 and 6 to phase 2; 7, 8 and 9 to phase 3; 10, 11 and 12 to phase 4; 13, 14 and 15 to phase 5; 16, 17 and 18 to phase −3; 19, 20 and 21 to phase −2; and 22, 23 and 24 to phase −1.

As shown in FIG. 24, the vector polar coordinate plane used for vector analysis may also be divided into 24 phases, and the functional state of the living body determined based upon distribution analysis of APR values. By this means, it becomes possible to determine the functional state of the living body in even more detail.

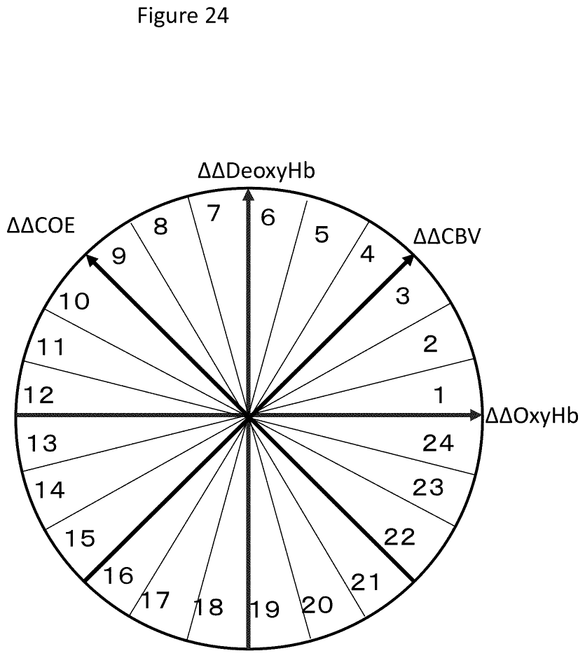

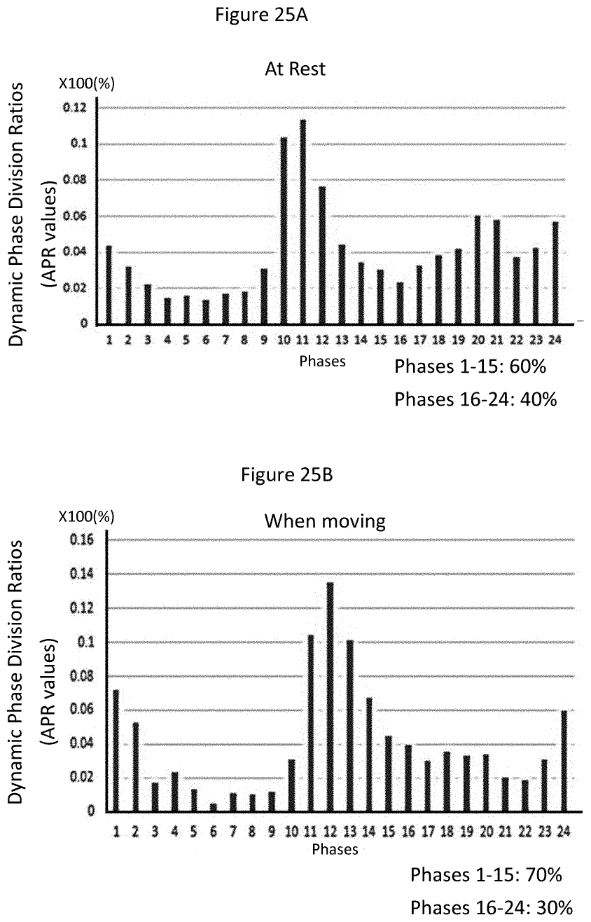

FIG. 25 is bar graphs in which the horizontal axis is phase (24 divisions) and the vertical axis is dynamic phase division ratios (APR values); 25A is the same data of a subject's state at rest from FIG. 14, where it was divided into 8 divisions, here divided into 24 divisions. 25B shows the case when the subject is moving.

When we compare FIG. 14 and FIG. 25A, we see that with respect to phase 4 of the 8 phase divisions, with 24 phase divisions, among phases divisions 10, 11 and 12, 11 has the highest frequency of occurrence. In this way, it is easier to tell a specific phase at rest, compared to with 8 phase divisions.

In cases of wide phase changes, such as between data at rest and in motion, more detailed tracking of phase changes is possible.

When we compare FIG. 25A and FIG. 25B, in comparison to 60% at rest for phases 1-15, and 40% for phases 16-24, when moving, phases 1-15 are 70% and phases 16-24 are 30%.

When moving, dynamic phase division ratios are increased by 10%; they are increased in phases 9-13, near the ΔΔCOE axis; and they are decreased at phases 6, 7, and 8 and 19-24. It can be determined that brain activity at ch4 is heightened by moving.

FIG. 26 are bar graphs in which the horizontal axes are phase (24 divisions) and the vertical axes are dynamic phase division ratios (APR values) at channels ch16, ch17 and ch18; 26A shows the subject at rest, and 26B shows the case when the subject is moving.

From a comparison of FIG. 26A and FIG. 26B, it can be determined that the APR values of phase 13 at ch18, at rest and when moving, changed the most.

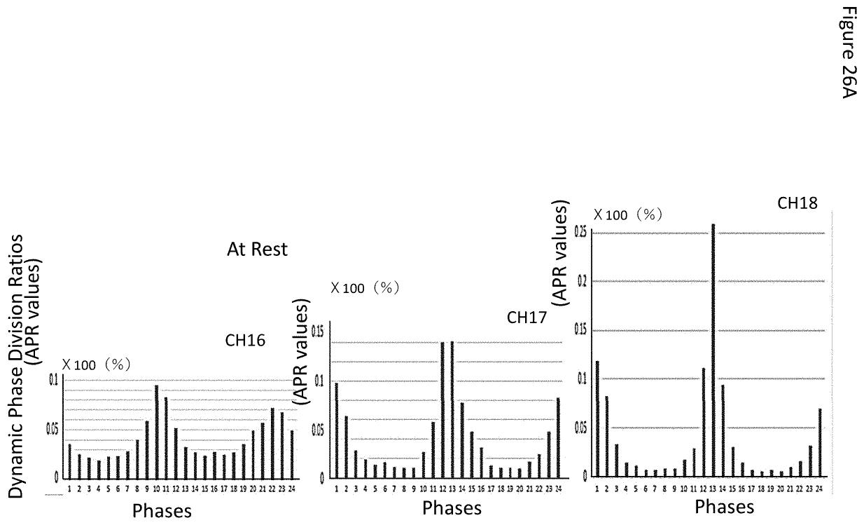

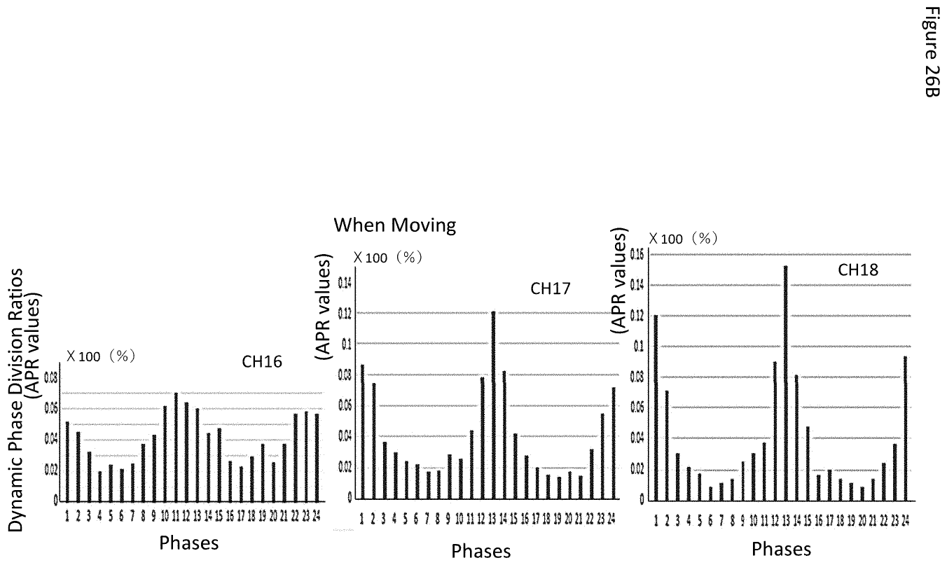

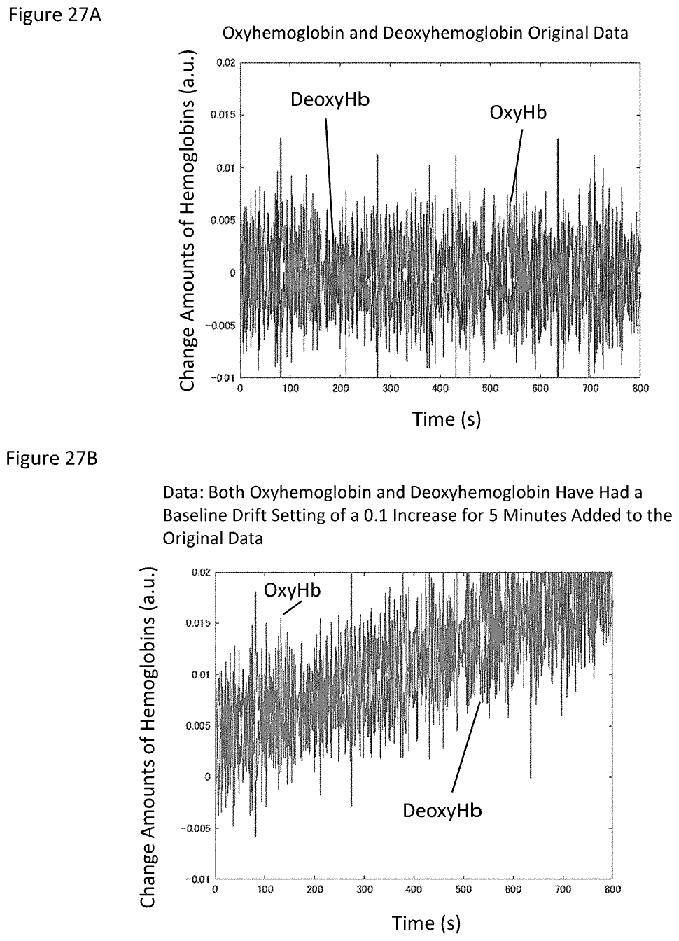

FIG. 27A shows a time course graph of original data obtained from channels of the frontal region at rest, displayed with the horizontal axis as time and the vertical axis as concentration change amounts of oxyhemoglobin and concentration change amounts of deoxyhemoglobin. 27B shows a time course graph in which a baseline drift setting of a 0.1 increase, continuing for 5 minutes, is added to both the oxyhemoglobin (OxyHb) and deoxyhemoglobin (DeoxyHb) of the original data of 27A, and displayed.

When we compute dynamic phase division ratios (APR values) from FIGS. 27A and 27B, the APR values of phases 1-5 have the identical values of 53.38%. Namely, with the use of zeroset vector groups, even if there is baseline drift, there is no change in the dynamic phase division ratios; and even if there is baseline drift, the state during time at rest can be effectively measured even without performing baseline correction.

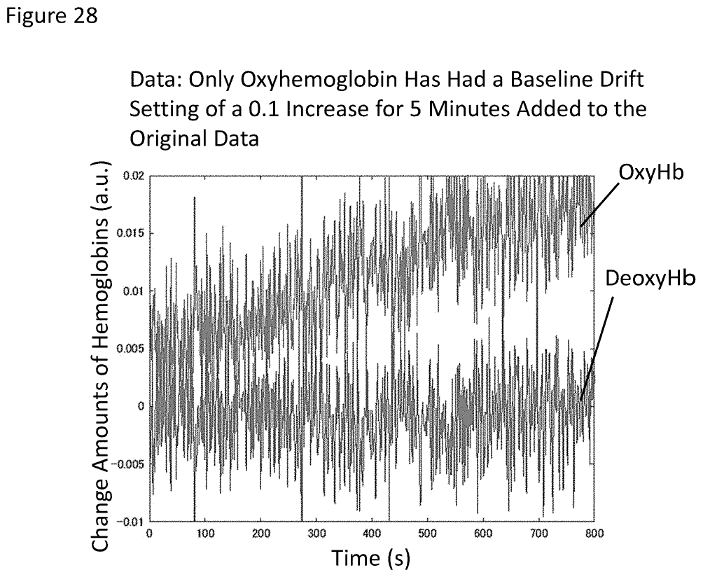

FIG. 28 shows a time course graph in which a baseline drift setting of a 0.1 increase, continuing for 5 minutes, is added only to the oxyhemoglobin (OxyHb) of the original data of FIG. 27A and displayed.

When dynamic phase division ratios were computed, the APR values of phases 1-5 showed 53.25%. Namely, in the case when the drift of only oxyhemoglobin is corrected, because of the fact that a 0.12% difference arises in the APR values, it is clear that the actual data is distorted.

Conventionally, in fNIRS analysis, analysis has been done with drift correction performed on the sole index of oxyhemoglobin alone, but it is clear that the precision of the data was distorted.

Effects of computing dynamic phase division ratios (APR values) are as follows:

(1) For example, even in the motor region, in sites related to movement of the mouth and oral cavity in the left brain and the right brain, brain activity could not be identified accurately by previous techniques, but by means of the use of dynamic phase division ratios, it is possible to quantify it and show its validity.

(2) In the midst of biting, indeed, dynamic phase division ratios (APR values) rise, and after biting is finished, they fall. The index APR is corresponding to this movement in real time.

From the fact that the APR increases even from at rest, the fact that low oxygenation or deoxygenation occurs, and that oxygen consumption occurred accompanying brain cell activity can be quantitatively determined. Namely, by means of the use of dynamic phase division ratios, the precision of brain activity detection in real time is improved.

(3) At rest, after biting is finished, APR values fall below their values before biting, but OxyHb (oxyhemoglobin) [values] are still increasing. By this means, it is possible to tell if it is a resting state, or a recovery state.

(4) With OxyHb, because its detection depends on the strength of changes, sensitivity to weak changes is low, but as for dynamic phase division ratios (APR values), because they utilize frequency of phase occurrence, they are not dependent on the strength of signal changes.

(5) Because there are percentage units for each person, with the conventional change amounts of OxyHb and change amounts of DeoxyHb, which used relative strengths, it has been difficult to compare individuals, do group analysis and the like, but these have become possible by the use of dynamic phase division ratios.

(6) It has become possible to detect the state of the brain at rest by classification into an arbitrary number of classifications, such as 8 classifications, 24 classifications, etc.

(7) By distinguishing time course changes of adjacent sites in real time, it has become possible to isolate time period[s] when brain activity is at its highest and so on, and even at rest, to detect and determine that the APR is increasing and brain activity is temporarily occurring.

(8)Even when there is baseline drift, true values can be obtained, without phases being distorted.

Conventionally, for fNIRS measurements, the main indices were change amounts of oxyhemoglobin and change amounts of deoxyhemoglobin, but by quantification using R and S, the use of dynamic phase division ratios, and so on, it is possible also to heighten the precision of brain-computer interface (BCI) and brain-machine interface (BMI).

Regarding the step for computing vector norms of the vector group (Step 5):

### Mathematical Expression 3

(ΔL)²=(ΔO)²+(ΔD)² (Equation 5)

(ΔΔL)²=(ΔΔO)²+(ΔΔD)² (Equation 7)

Utilizing equations (5) and (7), from subject data which measures radius distribution as a property of fundamental frequency, frequency distribution was computed for norms R (here called “radii”) of (ΔOxyHb, ΔDeoxyHb) and (ΔΔOxyHb, ΔΔDeoxyHb). Thinking that the frequency distribution of (ΔΔOxyHb, ΔΔDeoxyHb) was indicative of a radius distribution of the fundamental frequency, [the inventor] discovered properties showing a Rayleigh distribution.

FIG. 29A is a bar graph showing frequency distribution of radius R of (ΔOxyHb, ΔDeoxyHb) in subject A; 29B is a bar graph in which the frequency distribution of (ΔΔOxyHb, ΔΔDeoxyHb) shows a Rayleigh distribution.

FIG. 30A is a bar graph showing frequency distribution of radius R of (ΔOxyHb, ΔDeoxyHb) in subject B; 30B is a bar graph in which the frequency distribution of (ΔΔOxyHb, ΔΔDeoxyHb) shows a Rayleigh distribution.

Probability distribution of synthesized received signal electric field strengths of a multipath wave (sinusoidal wave), in which the wave frequency is constant and the amplitude and phase vary irregularly, follow a Rayleigh density distribution. When multipath waves arrive, from multiple refraction waves, duct propagation paths, and the like, and are synthesized, they follow this distribution. This distribution is used mainly in analysis of propagation paths in microwave radio communication, mobile wireless/radio communication, and the like.

This time, the inventor found that the transport path of oxygen in oxygen exchange has properties of wave function and follows a Rayleigh density distribution. By means of the properties of a Rayleigh density distribution, it is possible to distinguish oxygen exchange states at rest at different sites by computing maximum likelihood estimates.

This is clear from the differences of Rayleigh distributions of subjects A and B. What cannot be seen in ΔL distributions can be seen in ΔΔL distributions.

### Mathematical Expression 4

When the probability variable is taken to be a real number X (0≤X), the probability density function of the Rayleigh distribution is defined by the following equation.

$$\frac{x}{{σ}^{2}} exp ( - \frac{{x}^{2}}{2 {σ}^{2}} )$$

The expected value is

$$σ \sqrt{\frac{π}{2}}$$

and dispersion

$$( 2 - \frac{π}{2} ) {σ}^{2}$$

When obtained taking the observed value of the probability variable to be Xᵢ, the maximum likelihood estimate of the parameter is

$$\hat{σ} = \sqrt{\frac{1}{2 n} {\sum }_{i - 1}^{n} {x}^{2}}$$

FIG. 31A is a graph showing angular radius dispersion of (ΔOxyHb, ΔDeoxyHb); and 31B, a graph showing angular radius dispersion of (ΔΔOxyHb, ΔΔDeoxyHb). As a standard, they are displayed rotated 180 degrees in a counterclockwise direction or −180 degrees in a clockwise direction with respect to the ΔOxyHb axis or the ΔΔDeoxyHb axis. Because the zeroset vectors have an angle and a scalar value, distribution of both are displayed simultaneously.

From FIG. 31A, it can be seen that the angular radius distribution (ΔOxyHb, ΔDeoxyHb) of Subject C in a resting state tends to move largely 135 degrees in the direction of the COE axis. By this means, it can be seen that minute vector movements are increasing in a state nearly parallel to the CEO axis. Namely, it can be seen that movement that is not parallel to movement of the OxyHb, DeoxyHb, and CBV axes is a resting state.

When this is quantified, it can be determined by calculation of the equations shown in physiological property analysis example 5, by the fact that dispersion V is small (angle is biased).

On the other hand, from FIG. 31B, for (ΔΔOxyHb, ΔΔDeoxyHb), S (standard deviation) is small, at 0.1 or less, and thus the scalar values are relatively uniform, and their property of being distributed widely and evenly over 360 degrees became clear.

The differentiated angular radius distribution is uniform over 360 degrees, its anisotropy (directionality) is 360 degrees, and dispersion is high.

This in itself is surprising, and is a decisive new phenomenon by which time at rest can be physiologically defined.

Furthermore, in a case in which the directionality is biased, a state which varies from the state at rest, or a state in which external factors are included can be diagnosed.

### Mathematical Expression 5

Using angular/directional statistics, R, V, and S are computed

R=average vector norm L norm=L

V=1−R V is dispersion

S=\sqrt{−2log(R)}S is standard deviation

For example, data of subject D, at CH8 is resting state:

In the case of ( [symbol] OxyHb, [symbol] DeoxyHb):

R=0.967

V=0.033 Dispersion is small (angles are biased)

S=0.0172

In the case of ( [symbol] [symbol] OxyHb, [symbol] [symbol] DeoxyHb):

R=0.199

V=0.801 Dispersion is large (angles are scattered)

S=1.184

Namely, for large dispersion V (angles are scattered), S (standard deviation) is 1. As for large scalar values, of 1 and greater than 1, a comparatively large presence of differences can be seen.

FIGS. 32 are bar graphs in which the horizontal axes are channels (ch) and the vertical axes are dynamic phase division ratios (APR values), comparing data during 224 s at rest and during movement; 32A is norms R, from which APRs are created for the channels (at rest); 32B is norms R, from which APRs are created for the channels (during movement); 32C is standard deviations S of norms R, from which APRs are created for the channels (at rest); and 32D is standard deviation S of norms R, from which APRs are created for the channels (during movement).

At rest the amplitude of R is greater than during movement, and it is scattered. On the other hand, the standard deviation of R during movement is uniform.

Scattered at rest and becoming uniform during movement, can be evaluated as reflecting the fact that the brain is working for a specific goal, and like an orchestra, the brain is being caused to be active, continuously and in a constant direction.

In this way, by circular (angular) statistical values, computation of R and S makes it possible to quantitatively evaluate the state of the brain.

The advantage of analyzing data by standard deviations S, is that by evaluating not only dispersion, but also simultaneously S, indices for evaluating the physiological state at rest are increased, and precision is thus increased. Several combinations can be evaluated.

Digitization of large and small can also be set up arbitrarily, as shown below.

As examples:

V: 0-1

S: 0-1 SD, 2 SD, ≥3 SD

V: large (≥0.6), small (≥0.4), mid-range 0.4-0.6

S: large (SD≥2, small (SD≤1), mid-range 1<SD<2

Effects of using frequency distribution of norms of vectors of vector groups, circular (angular) statistics, and the like, to compute average vector norms, dispersion of norms L (V), and standard deviations (S) include those shown below.

(1) Stress states at rest can be evaluated. Furthermore, not simply can we quantify the strength/weakness of the stress, but detailed classification of the stress state also becomes possible, using phase distribution, circular (angular) statistics and the like to compute average vector norms (R), dispersion of norms L (V), and standard deviations (S) of those.

(2) When analysis is performed over prescribed intervals, not only average vector norms, dispersion (V) of norms L, and standard deviations (S) of those can be computed in the case of values of (ΔOxyHb, ΔDeoxyHb), but because analysis is also possible of average vector norms, dispersion (V) of norms L, and standard deviations (S) of those in the case of values of (ΔΔOxyHb, ΔΔDeoxyHb), compared to power spectrum analysis, even more detailed physiological differences of the resting state, and [differences between] at rest and during an activation task, and the like, can be distinguished.

(3) The state of the brain at rest can be quantitatively evaluated.

(4) Detailed classification of the state of the brain at rest is possible.

(5) Differentiation by means of quantification of the state in sleep, with eyes open, eyes closed, and the like is possible.

(6) From the state of the brain at rest, it is possible to quantify progressing stages of dementia, and to classify them.

### Mathematical Expression 6

Dual axial correlation coefficient of a rotating coordinate system (Generalized COE)

A point (x, y) of an X-Y coordinate system, rotated by angle θ around the origin is given the coordinates of point (g1, g2) in a rotating coordinate system G1-G2 of the following equation:

$$( \begin{matrix}g 1 \\ g 2\end{matrix} ) = ( \begin{matrix}x cos θ + y sin θ \\ - x sin θ + y cos θ\end{matrix} ) \qquad ( Equation 8 )$$

Coordinate system G₁−G₂ is called the GCOE(generalized COE) coordinate system of angle θ.

In the case when the coordinate rotation angle θ=45 degrees, the G₁ axis is the CBV axis and the G₂ axis is the COE axis.

A rotating coordinate system dual axis correlation graph showing the relationship between coordinate rotation angles and the dual axis correlation coefficients shows the coordinate rotational angle dependency of sample correlation coefficient (“cc” in the equation below, referred to below simply as “correlation coefficients”) of time course data on G₁ and G₂axes.

s_{G}_{₁}_{G}_{₂} is sample covariance; S_{G}_{₁} and are sample standard deviation of G₁ and G₂.

$$C C = \frac{{s}_{{G}_{1} {G}_{2}}}{{S}_{{G}_{1}} {S}_{{G}_{2}}} \qquad ( Equation 9 )$$

FIG. 33 is a graph showing a rotating coordinate system at rest (Generalized COE) for computing dual-axis correlation coefficients at the time of coordinate rotation angle θ using a zeroset vector group.

The table below is a table showing the relationship between the rotating coordinate system and dual-axis correlation coefficients.

TABLE 1
AS for positive/negative (+/−) of the correlations
coefficients between the data on the dual Generalized
COE(GCOE) axes G1 and G2 of coordinate rotation angle
θ, the table below shows which axes on the OxyHb-
DeoxyHb 2-dimensional coordinate system (the
correlation coefficients) are distributed
along (replaced by their opposite axis every 90 degrees.

| | Coordinate rotation angle θ | | | | | | | |
|---|---|---|---|---|---|---|---|---|
| | 0° | 45° | 90° | 135° | 180° | 225° | 270° | 315° |
| correlation | CBV | DeoxyHb | COE | OxyHb | CBV | DeoxyHb | COE | OxyHb |
| coefficients | | | | | | | | |
| + | | | | | | | | |
| correlation | COE | OxyHb | CBV | DeoxyHb | COE | OxyHb | CBV | DeoxyHb |
| coefficients | | | | | | | | |
| − | | | | | | | | |

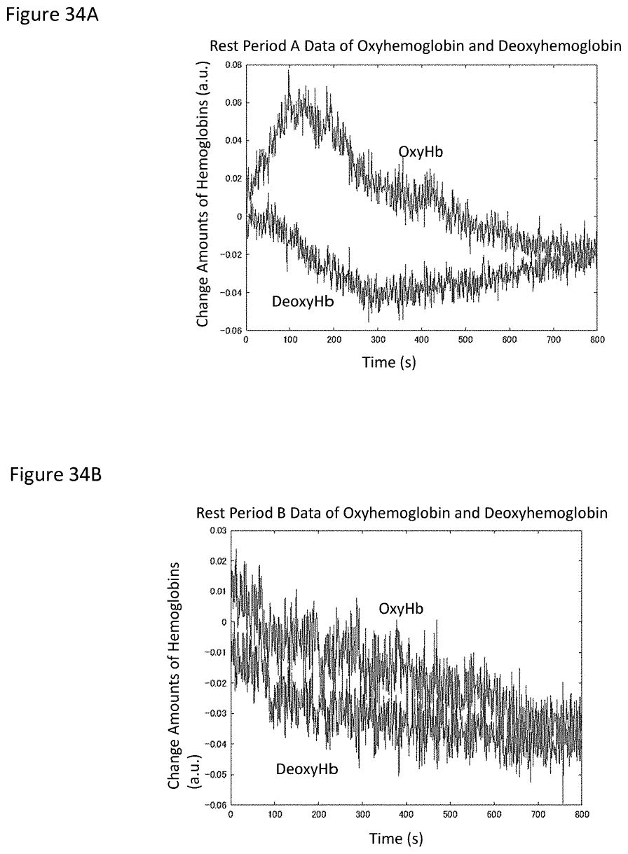

FIG. 34A is a time course graph obtained from a channel of the frontal region of Rest Period A, in which the horizontal axis is time, and the vertical axis is concentration change amounts of oxyhemoglobin and concentration changes of deoxyhemoglobin; FIG. 34B is a time course graph obtained from a channel in the frontal region of Rest Period B, in which the horizontal axis is time, and the vertical axis is concentration change amounts of oxyhemoglobin and concentration changes of deoxyhemoglobin.

The procedure for computing the rotating coordinate system dual-axis correlations from the rest period data of FIG. 34 are: from the time course graphs of concentration change amounts of oxyhemoglobin and concentration change amounts of deoxyhemoglobin shown in FIGS. 34 A and B, as shown in FIG. 7, a group of zeroset vectors is obtained at prescribed sampling times (for example,75 ms), based on 2-dimensional diagrams showing the relationship between the change amounts of oxyhemoglobin and the change amounts of deoxyhemoglobin (Step S2). Furthermore, the group of vectors is plotted on a 2-dimensional coordinate planes (Step S3).

Next, while rotating the Oxy-Deoxy coordinates on a 2-dimensional coordinate plane 360 degrees in a counterclockwise direction, computing correlation coefficients with the zeroset vector group plots (step S6).

On this occasion, the dual-axis plane created by coordinate rotation of an arbitrary angle of the plane is defined as GCOE. By means of differences of distribution of the plotted data, when rotating 360 degrees, the phases where correlations are highest and the phases where correlations are lowest are determined.

For example, in a case when the zeroset vectors are distributed in an oval shape, as in FIG. 33, correlation becomes higher at a coordinate rotation angle between ΔD and ΔCOE.

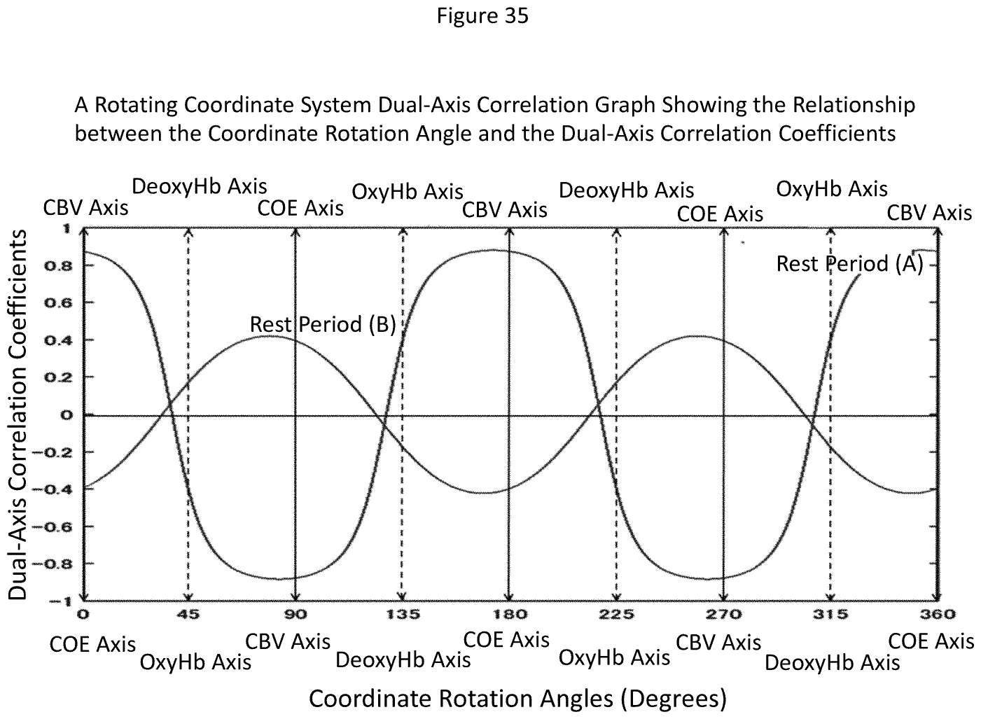

FIG. 35 is a rotating coordinate system dual-axis correlation graph of Rest Period A and Rest Period B, in which the horizontal axis is coordinate rotation angle (degrees) and the vertical axis is dual-axis correlation coefficients, and it shows the relationship of the two. The rotating coordinate system coincides with the OxyHb axis at coordinate rotation angles 0 degrees and 180 degrees, with the DeoxyHb axis at coordinate rotation angles 45 degrees and 225 degrees, with the COE axis at coordinate rotation angles 90 degrees and 270 degrees, with the OxyHb axis at coordinate rotation angles 135 degrees and 315 degrees, respectively. Because the correlation of the axes and the coordinate rotation angle is displayed on a rotating coordinate system dual-axis correlation graph, it is possible to tell instantly along which axes the zeroset vectors are distributed.

In fact, Rest Period A shows a high correlation of ≥+0.8 around 0 degrees and 180 degrees. It shows ≥−0.8 around 90 degrees and 270 degrees. Namely, if we refer to Table 1, the zeroset vector group of Rest Period A can be determined to be distributed showing a high correlation with the ΔCBV axis.

On the other hand, for Rest Period B, correlation is low in all the phases, but from the fact that it shows correlation coefficients of −0.4 around 0 degrees and 180 degrees, and correlation coefficients of +0.4 around 90 degrees and 270 degrees, it can be determined, with reference to Table 1, that the zeroset vectors of Rest Period B are distributed showing a high correlation to the ΔCOE axis, compared to the other 3 axes.

On the other hand, from the fact that both Rest Period A and Rest Period B show correlation coefficients of zero between 30 degrees and 45 degrees, between 120 degrees and 135 degrees, between 210 degrees and 225 degrees, and between 300 degrees and 315 degrees, it can be determined that there is no correlation with the ΔOxyHb axis and the ΔDeoxyHb axis.

In this way, it can be seen that the states in Rest Period A and Rest Period B are clearly different.

In this way, the computation of rotating coordinate system dual-axis correlation coefficients and the display and creation of a graph make possible an evaluation of the state at rest that cannot be obtained merely from time course data, and plotting [the data] on 2-dimensional coordinates, etc.

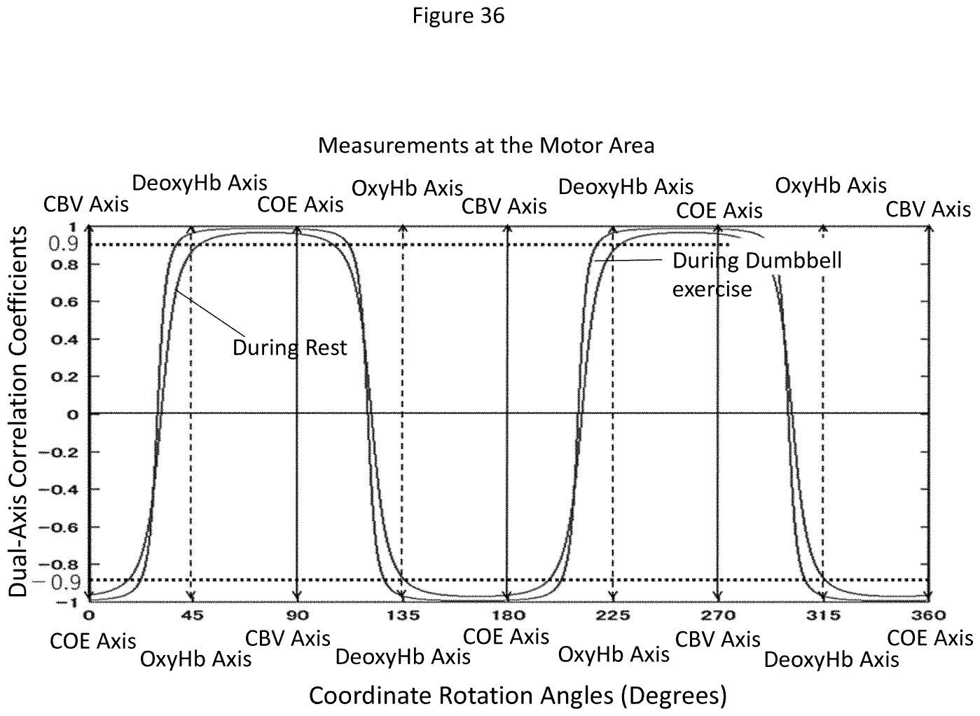

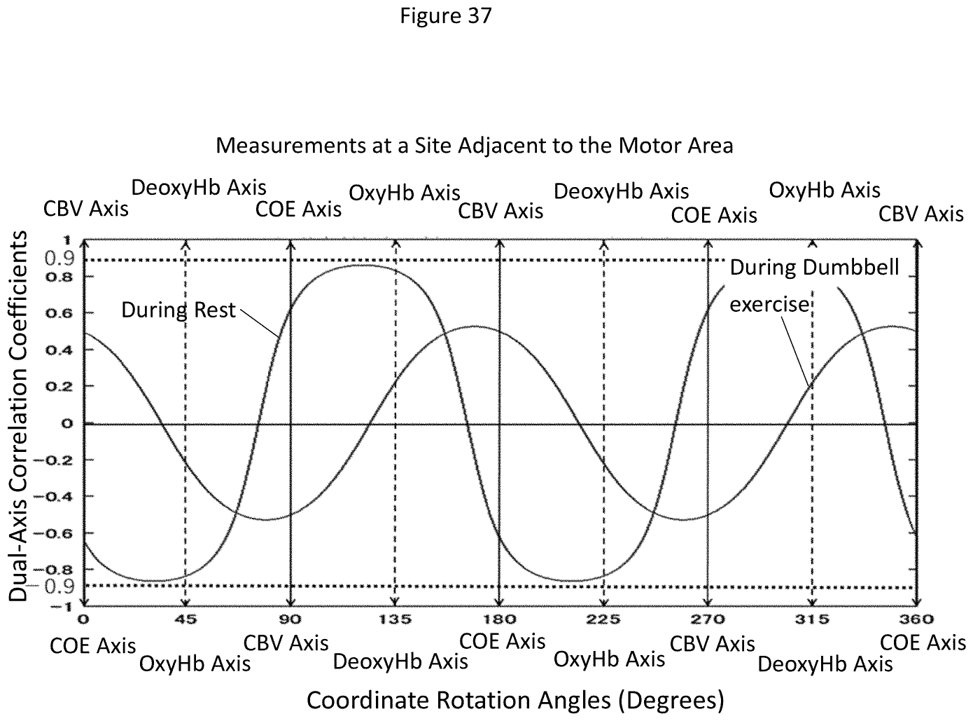

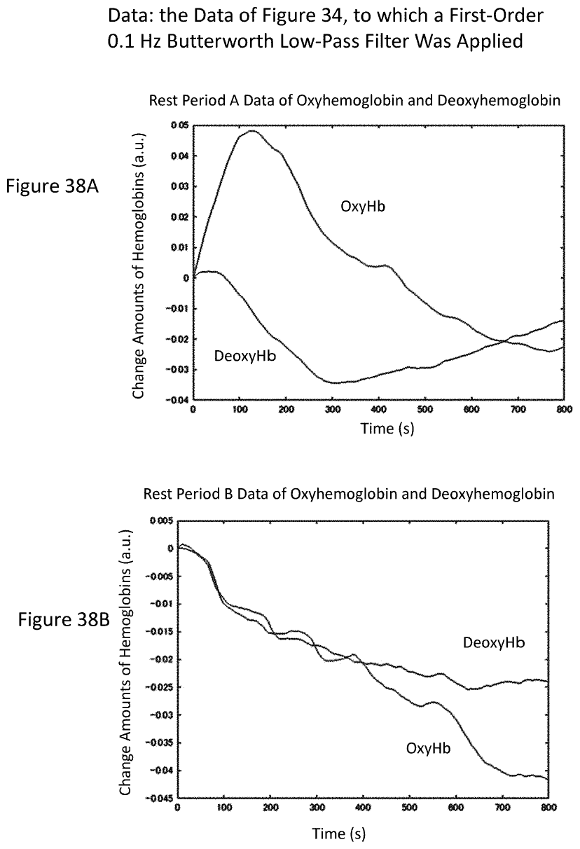

FIG. 36 is a rotating coordinate system dual-axis correlation graph of measured data obtained from the motor area (M1) during rest and during the exercise of lifting a 9.5 kg dumbbell. FIG. 37 is a rotating coordinate system dual-axis correlation graph of measured data obtained from a site adjacent to the motor area (M1) during rest and during the exercise of lifting a 9.5 kg dumbbell.

From FIG. 36, it can be seen that the zeroset vector groups during rest and during exercise are distributed overlapping one another in the axial directions between the DeoxyHb axis and the COE axis. The correlation coefficients show zero at coordinate rotation angles of 30 degrees, 120 degrees, 210 degrees and 300 degrees.

As for the coordinate rotation angles showing correlation coefficients of ≥0.9 and ≤−0.9, from the fact that they are over a wider range during the dumbbell exercise than during rest, it can be determined that it is a state of strong oxygen consumption, regulated to a high degree, without any to spare, for performing highly precise oxygen consumption. From the fact that correlation with coordinate rotation angles between the ΔOxyHb axis and the ΔCBV axis is low, it can be determined that in the motor region, the situation is not one in which oxygen is being supplied.

From FIG. 37, the zeroset vector group during rest shows correlation coefficients of ≥0.8 at 120 degrees and 300 degrees, and correlation coefficients of ≤−0.8 at 30 degrees and 210 degrees. It can be seen that correlation is distributed so that such a way that it is high at coordinate rotation angles between the ΔOxyHb axis and the ΔCBV axis. Namely, the coordinate rotation angles are around 45 degrees different, and at the site adjacent to the motor region (M1) during exercise, it can be determined to be a state in which oxygen consumption becomes high while oxygen supply is occurring, in comparison to the fact that the motor region correlates to the oxygen consumption axis.

On the other hand, from the fact that correlation of the zeroset vectors during the dumbbell exercise with coordinate rotation angles between the ΔOxyHb axis and the ΔCBV axis is high, at the site adjacent to the motor region (M1) it can be determined to be a situation in which oxygen supply is occurring, in contrast to the motor region, where oxygen consumption is occurring.

When the results during the dumbbell exercise at the motor region and at the site adjacent to the motor region are compared, phase differences of as much as 90 degrees are clearly perceived.

FIG. 38A is a graph in which the data of FIG. 34A, to which a first order 0.1 Hz Butterworth low-pass filter has been applied, is displayed, taking the horizontal axis to be time and the vertical axis to be concentration change amounts of oxyhemoglobin and concentration change amounts of deoxyhemoglobin.

FIG. 38B is a graph in which the data of FIG. 34B, to which a first order 0.1 Hz Butterworth low-pass filter has been applied, is displayed, taking the horizontal axis to be time and the vertical axis to be concentration change amounts of oxyhemoglobin and concentration change amounts of deoxyhemoglobin.

FIG. 39 is a rotating coordinate system dual-axis correlation graph of rest period (A) and rest period (B) showing the relationship of the dual-axis correlation coefficient to the coordinate rotation angle after low-pass filtering.

The results of FIG. 35 before low-pass filtering show a high correlation of the zeroset vector group of rest period (A) with the CBV axis, and show a high correlation of the zeroset vector group of rest period (B) with the ΔCOE axis.

However, after a first order 0.1 Hz low-pass filter was applied, rest period (A) shows correlation coefficients of ≥0.6 with the oxyhemoglobin axis. Rest period (B) shows correlation coefficients of ≥0.9 with an axis between the oxyhemoglobin axis and the CBV axis.

Namely, the phases changed after the low-pass filter: for the zeroset vector group axis of rest period (A), by approximately 45 degrees; and for the zeroset vector group axis of rest period (B), by approximately 180 degrees.

In this way, in the low frequency band of ≤0.1 Hz, two more changes in state became clear.

FIG. 40A is a graph displaying the data of FIG. 34A, to which a first order 0.1 Hz Butterworth high-pass filter was applied, taking the horizontal axis to be time and the vertical axis to be concentration change amounts of oxyhemoglobin and concentration change amounts of deoxyhemoglobin.

FIG. 40B is a graph displaying the data of FIG. 34B, to which a first order 0.1 Hz Butterworth high-pass filter was applied, taking the horizontal axis to be time and the vertical axis to be concentration change amounts of oxyhemoglobin and concentration change amounts of deoxyhemoglobin.

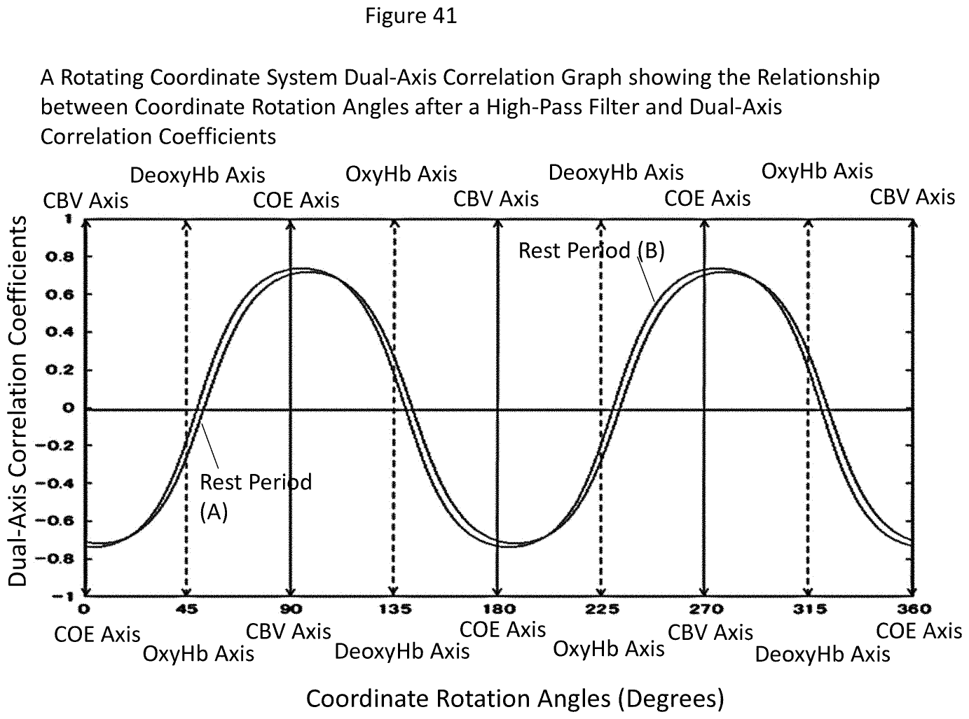

FIG. 41 is a rotating coordinate system dual-axis correlation graph of coordinate rotation angles of Rest Period A and Rest Period B, showing the relationship of dual-axis correlation coefficients to coordinate rotation angles after a high pass filter.

The zeroset vector groups of Rest Period A and Rest Period B each show a high correlation with the COE axis; and the phases changed after the high-pass filter: by approximately 30 degrees for the zeroset vector group axis of rest period (A); and by approximately 90 degrees for the zeroset vector group axis of rest period (B).

It can be determined that the fact that the high frequency bands of the zeroset vector groups of Rest Period A and Rest Period B approach [each other] extremely closely is clear.

In the high frequency band, which has until now been considered to be a noise component and has been removed by means of a filter prior to analysis processing, zeroset vector groups of Rest Period A and Rest Period B show a high correlation with the COE axis, but it has become clear that a component of neural activity is contained [therein].

From FIG. 39 and FIG. 41, from the fact that the shifts of phase of the zeroset vector groups of Rest Period A and Rest Period B differ after high-pass filtering and low-pass filtering, filtering by different frequency bands and [then] computation of coordinate rotation angle dependence from dual-axis correlation coefficients is an effective way to quantifying the state at rest.

Effects of computation of dual-axis correlations of a rotating coordinate system are as follows:

(1) The state of oxygen metabolism at rest, such as oxygen consumption, oxygen supply, blood flow increase, and blood flow decrease, can be quantified by site.

(2) The state of oxygen metabolism at rest can be compared quantitatively among sites.

(3) The state of oxygen metabolism at rest can be compared quantitatively among individuals.

(4) If a group of zeroset vectors can be created, a rest period can be quantitatively evaluated, within a short period of time.

(5) Quantification at rest by [different] frequency bands is possible.

(6) Sensitivity and precision of comparisons is improved. during rest and during tasks, between individuals, between tasks, and by site.

(7) Changes of the state at rest can be quantified by phase.

### Program

Program 11 of the embodiment of the present invention shown in FIG. 1 is a program 11 that implements processing of the apparatus for evaluating biological function, performed by means of said apparatus for evaluating biological function.

This Program 11 may be saved on memory media such as a magnetic disk, CD-ROM, semiconductor memory, etc.; it may also be downloaded over a communications network.

The present invention is not limited to the embodiment described above, and within the range of the technical matters in claims, various changes are possible.

### EXPLANATION OF THE SYMBOLS

1: Apparatus for evaluating biological function

2: Light-emitting part

3: Light-receiving part

4: Detection part

5: Measuring part

6: Determination part

7: Display part

8: Computing part

9: Memory part

10: Image processing part

11: Program

1. An apparatus for evaluating biological function, which evaluates biological function utilizing the near-infrared spectroscopy method, having
a plurality of detecting parts provided with light-emitting parts for irradiating light to a prescribed site of a living body and light-receiving parts for receiving and detecting light emanating from the living body;
a measuring part, into which light information detected by means of said detecting parts is input, and wherein computation, control and/or memory operations are performed; and
a determination part for determining the state of biological function of said living body;
wherein said measuring part has a computing means for computing time course change amounts of oxyhemoglobin and time course change amounts of deoxyhemoglobin based on light information from said detecting parts, obtaining a vector group zero-set at prescribed sampling times based on 2-dimensional diagrams showing the relationship between said change amounts of oxyhemoglobin and change amounts of deoxyhemoglobin, and computing parameters based on the directions and scalars of said vector group; and
said determination part determines the state of biological function based on said parameters computed by means of said computing means.

2. The apparatus for evaluating biological function according to claim 1, characterized in that said parameters are dynamic phase division ratios, based on said 2-dimensional diagrams, for showing how frequently said vector group vectors appear in the plurality of phase divisions into which they are divided.

3. The apparatus for evaluating biological function according to claim 1, characterized in that said parameters are parameters concerning norms of vectors of said vector group.

4. The apparatus for evaluating biological function according to claim 3, characterized in that said parameters are any or all of: average vector norms, dispersions of norms, standard deviations of norms, of the vectors of said vector group, computed using circular statistics angular statistics).

5. The apparatus for evaluating biological function according to claim 1, characterized in that said parameters are parameters computed based on a probability density function of a Rayleigh distribution.

6. The apparatus for evaluating biological function according to claim 1, characterized in that said parameters are parameters concerning correlation properties of the 2 orthogonal axial directions on said 2-dimensional diagrams.

7. The apparatus for evaluating biological function according to claim 1, characterized in that said parameters are parameters concerning a rotating coordinate system using said vector group.

8. The apparatus for evaluating biological function according to claim 1, characterized in that said parameters are parameters concerning correlation coefficients of 2 orthogonal axial directions on said 2-dimensional diagrams with coordinate rotation angle[s] on a rotating coordinate system using said vector group.

9. The apparatus for evaluating biological function according to claim 1, characterized in that said parameters are parameters computed using derivatives of said time course change amounts of oxyhemoglobin and time course change amounts of deoxyhemoglobin.

10. The apparatus for evaluating biological function according to claim 1, characterized in that said parameters are parameters computed using derivatives of said time course change amounts of oxyhemoglobin and time course change amounts of deoxyhemoglobin, differentiated a plurality of times.

11. The apparatus for evaluating biological function according to claim 1, characterized in that said parameters are parameters computed varying said sampling times.

12. The apparatus for evaluating biological function according to claim 1, characterized in that said parameters are parameters computed selecting unit width numbers which are said sampling time multiplied by n (where n is an arbitrary number such that n>1).

13. The apparatus for evaluating biological function according to claim 1, characterized in that the values of said parameters are displayed on the display part, as data plotted on said 2-dimensional diagrams.

14. The apparatus for evaluating biological function according to claim 1, characterized in that said determination part takes the period wherein said task for activating brain function is not assigned to said living body to be brain time at rest, and determines the state of said biological function of that time at rest.

15. The apparatus for evaluating biological function according to claim 2, characterized in that said phase division diagrams are divided into eight divisions.

16. The apparatus for evaluating biological function according to claim 2, characterized in that said phase division diagrams are divided into 24 divisions.

17. A method for evaluating biological function that is performed by an apparatus for evaluating biological function, which evaluates biological function utilizing the near-infrared spectroscopy method, having a plurality of detecting parts provided with light-emitting parts for irradiating light to a prescribed site of a living body and light-receiving parts for receiving and detecting light emanating from the living body; a measuring part, into which light information detected by means of said detecting parts is input, and wherein computation, control and/or memory operations are performed; and a determination part for determining the state of biological function of said living body; comprising:
a step whereby time course change amounts of oxyhemoglobin and time course change amounts of deoxyhemoglobin are computed based on light information from said detecting parts;
a step whereby a group of vectors zero-set for each prescribed sampling time is obtained based on 2-dimensional diagrams showing the relationship between said change amounts of oxyhemoglobin and change amounts of deoxyhemoglobin, and parameters are computed based on the directions and scalars of said vector group, and
a step whereby the state of biological function is determined based on said computed parameters.

18. A program for implementing a biological function evaluation process performed by means of an apparatus for evaluating biological function, which evaluates biological function utilizing the near-infrared spectroscopy method, having a plurality of detecting parts provided with light-emitting parts for irradiating light to a prescribed site of a living body and light-receiving parts for receiving and detecting light emanating from the living body; a measuring part, into which light information detected by means of said detecting parts is input, and wherein computation, control and/or memory operations are performed; and a determination part for determining the state of biological function of said living body; comprising:
a process whereby time course change amounts of oxyhemoglobin and time course change amounts of deoxyhemoglobin are computed, based on light information from said detecting part;
a processes for obtaining a group of vectors zero-set for each prescribed sampling time based on 2-dimensional diagrams showing the relationship between said change amounts of oxyhemoglobin and change amounts of deoxyhemoglobin, and for computing parameters based on the directions and scalars of said vector group, and
a process for determining the state of biological function based on said computed parameters.
